(19) United States
(12) Patent Application Publication
(10) Pub. No.: US 2005/0152588 A1
(43) Pub. Date: Jul. 14, 2005

(54) Method for virtual endoscopic visualization of the colon by shape-scale signatures, centerlining, and computerized detection of masses

(75) Inventors: Hiroyuki Yoshida, Chicago, IL (US); Janne J. Nappi, Chicago, IL (US); Hans Frimmel, Uppsala (SE); Abraham H. Dachman, Chicago, IL (US)

Correspondence Address: OBLON, SPIVAK, MCCLELLAND, MAIER & NEUSTADT, P.C., 1940 DUKE STREET, ALEXANDRIA, VA, 22314, US

(73) Assignee: University of Chicago, Chicago, IL (US)

(21) Appl. No.: 10/974,699

(22) Filed: Oct. 28, 2004

Related U.S. Application Data
(60) Provisional application No. 60/514,599, filed on Oct. 28, 2003.

(51) Int. Cl. G06K 9/00; G06K 9/46; G06K 9/66; A61B 5/05

(52) U.S. Cl. 382/128; 600/407; 382/190; 382/154; 382/162

(57) ABSTRACT

A visualization method and system for virtual endoscopic examination of CT colonographic data by use of shape-scale analysis. The method provides each colonic structure of interest with a unique color, thereby facilitating rapid diagnosis of the colon. Two shape features, called the local shape index and curvedness, are used for defining the shape-scale spectrum. The shape index and curvedness values within CT colonographic data are mapped to the shape-scale spectrum in which specific types of colonic structures are represented by unique characteristic signatures in the spectrum. The characteristic signatures of specific types of lesions can be determined by use of computer-simulated lesions or by use of clinical data sets subjected to a computerized detection scheme. The signatures are used for defining a 2-D color map by assignment of a unique color to each signature region.

### CROSS-REFERENCE TO RELATED APPLICATIONS

The present application is related to U.S. Patent Application Ser. No. 60/514,599, filed Oct. 28, 2003, and U.S. patent application Ser. No. 10/270,674, filed Oct. 16, 2002, the contents of which are incorporated herein by reference.

### STATEMENT REGARDING FEDERALLY SPONSORED RESEARCH

The present invention was made in part with U.S. Government support under USPHS Grant No. CA095279. The U.S. Government may have certain rights to this invention.

### BACKGROUND OF THE INVENTION

Field of the Invention

The present invention relates generally to systems and methods for the computer-aided detection of three-dimensionally extended organ lesions. The present invention also generally relates to automated techniques for the detection of abnormal anatomic regions, for example, as disclosed, in particular, in one or more of U.S. Pat. Nos. 4,907,156; 5,133,020; 5,832,103; and 6,138,045; all of which are incorporated herein by reference. Further, the present invention relates to visualization methods for virtual endoscopic examination of CT colonographic data.

The present invention also generally relates to computerized techniques for the automated analysis of digital images, for example, as disclosed in one or more of U.S. Pat. Nos. 4,839,807; 4,841,555; 4,851,984; 4,875,165; 4,907,156; 4,918,534; 5,072,384; 5,133,020; 5,150,292; 5,224,177; 5,289,374; 5,319,549; 5,343,390; 5,359,513; 5,452,367; 5,463,548; 5,491,627; 5,537,485; 5,598,481; 5,622,171; 5,638,458; 5,657,362; 5,666,434; 5,673,332; 5,668,888; 5,732,697; 5,740,268; 5,790,690; 5,832,103; 5,873,824; 5,881,124; 5,931,780; 5,974,165; 5,982,915; 5,984,870; 5,987,345; 6,011,862; 6,058,322; 6,067,373; 6,075,878; 6,078,680; 6,088,473; 6,112,112; 6,138,045; 6,141,437; 6,185,320; 6,205,348; 6,240,201; 6,282,305; 6,282,307; 6,317,617; 6,335,980; 6,363,163; 6,442,287; 6,466,689; 6,470,092; 6,594,378; 6,738,499; 6,754,380; 6,813,375; as well as U.S. patent application Ser. Nos. 09/759,333; 09/760,854; 09/773,636; 09/816,217; 09/830,562; 10/120,420; 10/270,674; 10/292,625; 10/358,337; 10/360,814; all of which are incorporated herein by reference.

The present invention includes use of various technologies referenced and described in the above-noted U.S. Patents and Applications, as well as described in the references identified in the following LIST OF REFERENCES by the author(s) and year of publication, and cross-referenced throughout the specification by reference to the respective number, in brackets, of the reference:

### LIST OF REFERENCES

[1] S. Winawer, R. Fletcher, L. Miller, F. Godlee, M. Stolar, C. Mulrow, S. Woolf, S. Glick, T. Ganiats, J. Bond, L. Rosen, J. Zapka, S. Olsen, F. Giardiello, J. Sisk, R. Van Antwerp, C. Brown-Davis, D. Marciniak, and R. Mayer, “Colorectal cancer screening: clinical guidelines and rationale,” Gastroenterology, vol. 112, pp. 594-642, 1997.
[2] A. Dachman, Ed., Atlas of Virtual Colonoscopy. New York, USA: Springer-Verlag, 2003.
[3] D. Vining, “Virtual colonoscopy,” Semin Ultrasound CT MR, vol. 20, pp. 56-60, 1999.
[4] C. Johnson and A. Dachman, “CT colonography: the next colon screening examination?” Radiology, vol. 216, pp. 331-341, 2000.
[5] J. Ferrucci, “Virtual colonoscopy for colon cancer screening: Further reflection on polyps and politics,” Am J Roentgenol, vol. 181, pp. 795-797, 2003.
[6] A. Royster, H. Fenlon, P. Clarke, D. Nunes, and J. Ferrucci, “CT colonoscopy of colorectal neoplasms: two-dimensional and three-dimensional virtual-reality techniques with colonoscopic correlation,” Am J Roentgenol, vol. 169, pp. 1237-1242, 1997.
[7] A. Sonnenberg, F. Delco, and J. Inadomi, “Cost-effectiveness of colonoscopy in screening for colorectal cancer,” Ann Intern Med, vol. 133, pp. 573-584, 2000.
[8] A. Dachman, “Diagnostic performance of virtual colonoscopy,” Abdom Imaging, vol. 27, pp. 260-267, 2002.
[9] C. Johnson, W. Harmsen, L. Wilson, R. MacCarty, T. Welch, D. Ilstrup, and D. Ahlquist, “Prospective blinded evaluation of computed tomographic colonography for screen detection of colorectal polyps,” Gastroenterology, vol. 125, pp. 311-319, 2003.
[10] E. McFarland, J. Brink, J. Loh, G. Wang, V. Argiro, D. Balfe, J. Heiken, and M. Vannier, “Visualization of colorectal polyps with spiral CT colography: evaluation of processing parameters with perspective volume rendering,” Radiology, vol. 205, pp. 701-707, 1997.
[11] C. Beaulieu, D. Paik, S. Napel, and R. Jeffrey Jr., “Advanced 3D display methods,” in Atlas of Virtual Colonoscopy, A. Dachman, Ed. New York, USA: Springer-Verlag, 2003.
[12] G. Rubin, C. Beaulieu, V. Argiro, H. Ringl, A. Norbash, J. Feller, M. Dake, R. Jeffrey, and S. Napel, “Perspective volume rendering of CT and MR images: applications for endoscopic imaging,” Radiology, vol. 199, pp. 321-330, 1996.
[13] S. Dave, G. Wang, B. Brown, E. McFarland, Z. Zhang, and M. Vannier, “Straightening the colon with curved cross sections: an approach to CT colonography,” Acad Radiol, vol. 6, pp. 398-410, 1999.
[14] S. Haker, S. Angenent, A. Tannenbaum, and R. Kikinis, “Nondistorting flattening maps and the 3-D visualization of colon CT images,” IEEE Trans Med Imaging, vol. 19, pp. 665-670, 2000.
[15] K. Mori, Y. Hayashi, J. Toriwaki, J. Hasegawa, and K. Katada, “A method for detecting unobserved regions in a virtual endoscopy system,” in SPIE Medical Imaging, vol. 4321, 2001, pp. 134-145.
[16] P. Rogalla, P. Bender, U. Bick, A. Huitema, J. Terwisscha van Scheltinga, and B. Hamm, “Tissue transition project (TTP) of the intestines,” Eur Radiol, vol. 10, pp. 806-810, 2000.
[17] J. Russ, The image processing handbook. CRC Press, 1994.
[18] A. Weeks Jr., Fundamentals of electronic image processing. SPIE, IEEE Press, 1996.
[19] J. Choi, M. Morin, R. Farrel, J. Potter, J. Kruskal, and V. Raptopoulos, “Color spectral imaging (CSI)-enhanced 3-D multi-planar reformation (MPR) for the evaluation of colonic pathology: interactive comparison with conventional colonoscopy and CT colonography,” Radiology, vol. 213(P), p. 496, 1999.
[20] R. Summers, C. Beaulieu, L. Pusanik, J. Malley, R. J. Jeffrey, D. Glazer, and S. Napel, “Automated polyp detector for CT colonography: feasibility study,” Radiology, vol. 216, pp. 284-290, 2000.
[21] M. Wax, K. Hopper, and K. Kreeger, “Translucent volume rendering for virtual colonoscopy,” Radiology, vol. 225(P), p. 739, 2002.
[22] H. Yoshida, J. Näppi, and A. Dachman, “Computer-aided visualization and differentiation of stool from polyps in CT colonography,” Radiology, vol. 221(P), p. 653, 2001.
[23] A. Dachman, R. Shah, J. Näppi, and H. Yoshida, “Three-dimensional color representation of CT colonography for differentiation of polyps from stool,” in Proc Third International Symposium for Virtual Colonoscopy, 2002, p. 130.
[24] A. Hilton, J. Illingworth, and T. Windeatt, “Statistics of surface curvature estimates,” Pattern Recognition, vol. 28(8), pp. 1201-1221, 1995.
[25] J.-P. Thirion and A. Gourdon, “Computing the differential characteristics of isointensity surfaces,” Computer Vision and Image Understanding, vol. 61, pp. 190-202, 1995.
[26] O. Monga and S. Benayoun, “Using partial derivatives of 3D images to extract typical surface features,” Computer Vision and Image Understanding, vol. 61, pp. 171-189, 1995.
[27] S. Kobayashi and K. Nomizu, Foundations of differential geometry.Interscience Press, 1963, vol. 1.
[28] S. Kobayashi and K. Nomizu, Foundations of differential geometry.Interscience Press, 1969, vol. 2.
[29] J. Koenderink, Solid shape.Cambridge, USA: MIT Press, 1990.
[30] P. Vaidyanathan, Multirate systems and filter banks.USA: Prentice Hall, 1992.
[31] M. Vetterli and J. Kovacevic, Wavelets and subband coding.USA: Prentice Hall, 1995.
[32] T. Lindeberg, Scale-scape theory in computer vision.Boston, USA: Kluwer Academic Publishers, 1993.
[33] H. Yoshida and J. Näppi, “Three-dimensional computer-aided diagnosis scheme for detection of colonic polyps,” IEEE Trans Med Imaging, vol. 20, pp. 1261-1274, 2001.
[34] H. Yoshida, J. Näppi, P. MacEneaney, D. Rubin, and A. Dachman, “Computer-aided diagnosis scheme for detection of polyps at CT colonography,” Radiographics, vol. 22, pp. 963-979, 2002.
[35] J. Näppi and H. Yoshida, “Feature-guided analysis for reduction of false positives in CAD of polyps for CT colonography,” Med Phys, vol. 30, pp. 1592-1601, 2003.
[36] J. Näppi, P. MacEneaney, A. Dachman, and H. Yoshida, “Knowledge-guided automated segmentation of colon for computer-aided detection of polyps in CT colonography,” J Comput Assist Tomogr, vol. 26, pp. 493-504, 2002.
[37] A. Watt, Fundamentals of three-dimensional computer graphics.USA: Addison-Wesley, 1989.
[38] C. Metz, Handbook of Medical Imaging vol 1: Medical Physics and Psychophysics.SPIE, 2001, ch. Fundamental ROC Analysis, pp. 751-769.
[39] T. Kobayashi, X. Xu, H. MacMahon, C. Metz, and K. Doi, “Effect of a computer-aided diagnosis scheme on radiologists' performance in detection of lung nodules on radiographs,” Radiology, vol. 199, pp. 843-848, 1996.
[40] Y. Jiang, R. Nishikawa, R. Schmidt, A. Toledano, and K. Doi, “Potential of computer-aided diagnosis to reduce variability in radiologists' interpretations of mammograms depicting microcalcifications,” Radiology, vol. 220, pp. 787-794, 2001.
[41] M J O'Brien, S J Winawer, A G Zauber, L S Gottlieb, S S Sternberg, B Diaz, G R Dickersin, S Ewing, S Geller, and D Kasimian et al. The national polyp study: patient and polyp characteristics associated with high-grade dysplasia in colorectal adenomas. Gastroenterology, 98: 371-379, 1990.
[42] J D Potter, M L Slattery, R M Bostick, and S M Gapstur. Colon cancer: a review of the epidemiology. Epidemiol Rev, 15: 499-545, 1993.
[43] S J Winawer, A G Zauber, M N Ho, M J O'Brien, L S Gottlieb, S S Stemberg, J D Waye, M Schapiro, J H Bond, and J F Panish et al. Prevention of colorectal cancer by colonoscopic polypectomy. The National Polyp Study Workgroup. N Engl J Med, 329: 1977-1981, 1993.
[44] J Yee and E McFarland. How accurate is CT colonography? In A H Dachman, editor, Atlas of Virtual Colonoscopy, New York, USA, 2003. Springer-Verlag.
[45] D J Vining, Y Ge, D K Ahn, and D R Stelts. Virtual colonoscopy with computer-assisted polyp detection. In K. Doi, H. MacMahon, M. L. Giger, and K. R. Hoffmann, editors, Computer-Aided Diagnosis in Medical Imaging, pages 445-452, Amsterdam, 1999. Elsevier Science.
[46] R M Summers, CD Johnson, L M Pusanik, J D Malley, A M Youssef, and J E Reed. Automated polyp detection at CT colonography: feasibility assessment in a human population. Radiology, 219: 51-59, 2001.
[47] DS Paik, C F Beaulieu, RB Jeffrey, J Yee, A M Steinauer-Gebauer, and S Napel. Computer aided detection of polyps in CT colonography: method and free-response. ROC evaluation of performance. Radiology, 2000. 217(P): 370.
[48] DS Paik, C F Beaulieu, A Mani, R W Prokesch, J Yee, and S Napel. Evaluation of computer-aided detection in CT colonography: potential applicability to a screening population. Radiology, 221(P): 332, 2001.
[49] H Yoshida, Y Masutani, P MacEneaney, D T Rubin, and A H Dachman. Computerized detection of colonic polyps at CT colonography on the basis of volumetric features: a pilot study. Radiology, 222: 327-336, 2002.
[50] G Kiss, J Van Cleynenbreugel, M Thomeer, P Suetens, and G Marchal. Computer-aided diagnosis in virtual colonography via combination of surface normal and sphere fitting methods. European Radiology, 12: 77-81, 2002.
[51] S B Göktürk, C Tomasi, B Acar, C F Beaulieu, and D Paik et al. A statistical 3-D pattern processing method for computer-aided detection of polyps in CT colonography. IEEE Trans Med Imaging, 20: 1251-1260, 2001.
[52] J Näppi and H Yoshida. Automated detection of polyps in CT colonography: evaluation of volumetric features for reduction of false positives. Acad Radiol, 9: 386-397, 2002.
[53] B Acar, C F Beaulieu, S B Göktürk, C Tomasi, and D Paik et al. Edge displacement field based-classification for improved detection of polyps in CT colonography. IEEE Trans Med Imaging, 21: 1461-1467, 2002.
[54] A K Jerebko, J D Malley, M Franaszek, and R M Summers. Multiple neural network classification scheme for detection of colonic polyps in ct colonography data sets. Acad Radiol, 10: 154-160, 2003.
[55] M Morrin, J Sosna, J Kruskal, A Blachard, N Stanietzky, and B Siewert. Diagnostic performance of radiologists with differing levels of expertise in the evaluation of CT colonography. Radiology, 223(P): 365, 2003.
[56] J Näppi, H Frimmel, A H Dachman, and H Yoshida. Computer-aided detection of masses in CT colonography: techniques and evaluation. Radiology, 225(P): 405, 2002.
[57] G Lohmann. Volumetric image analysis. John Wiley & Sons, New York, 1998.
[58] C-T Lin and C S G Lee. Neural fuzzy systems: a neuro-fuzzy synergism to intelligent systems. Prentice-Hall, New Jersey, USA, 1996.
[59] JA Sethian. Level Set Methods and Fast Marching Methods. Cambridge University Press, USA, 1999.
[60] G D Tourassi and C E Floyd. The effect of data sampling on the performance evaluation of artificial neural networks in medical diagnosis. Med Decis Making, 17: 186-192, 1997.
[61] C E Metz. Evaluation of CAD methods. In K. Doi et al., editor, Computer-Aided Diagnosis in Medical Imaging, pages 543-554. Elsevier Science, 1999.
[62] A H Dachman, J Näppi, H Frimmel, and H Yoshida. Sources of false positives in computerized detection of polyps in CT colonography. Radiology, 225(P): 303, 2002.
[63] ACS. Cancer facts and figures. American Cancer Society 2003.
[64] Vining DJ. Virtual colonoscopy. Gastrointest Endosc Clin N Am 1997; 7: 285-291.
[65] Ferrucci JT. Colon cancer screening with virtual colonoscopy: promise, polyps, politics. AJR Am J Roentgenol 2001; 177: 975-988.
[66] McFarland E G. Reader strategies for CT colonography. Abdom Imaging 2002; 27: 275-283.
[67] Zhang Z, Wang G, Brown B P, McFarland E G, Haller J, Vannier M W. Fast algorithm for soft straightening of the colon. Acad Radiol 2000; 7: 142-148.
[68] Paik D S, Beaulieu C F, Jeffrey R B, Jr., Karadi C A, Napel S. Visualization modes for CT colonography using cylindrical and planar map projections. J Comput Assist Tomogr 2000; 24: 179-188.
[69] Ge Y, Stelts D R, Wang J, Vining DJ. Computing the centerline of a colon: a robust and efficient method based on 3D skeletons. J Comput Assist Tomogr 1999; 23: 786-794.
[70] Ge Y, Stelts D R, Wang J, Vining DJ. Computing the central path of colon lumen in helical CT images. In:SPIE Medical Imaging, 1998; 702-713.
[71] Paik D S, Beaulieu C F, Jeffrey R B, Rubin G D, Napel S. Automated flight path planning for virtual endoscopy. Med Phys 1998; 25: 629-637.
[72] Samara Y, Fiebich M, Dachman A H, Kuniyoshi J K, Doi K, Hoffmann K R. Automated calculation of the centerline of the human colon on CT images. Acad Radiol 1999; 6: 352-359.
[73] Wan M, Dachille F, Kaufman A. Distance-field based skeletons for virtual navigation. In: 12th IEEE Visualization 2001 Conference. San Diego, Calif., 2001.
[74] Bitter I, Kaufman A E, Sato M. Penalized-distance volumetric skeleton algorithm. IEEE Transactions on Visualization and Computer Graphics 2001; 7: 195-206.
[75] Deschamps T, Cohen L D. Fast extraction of minimal paths in 3D images and applications to virtual endoscopy. Med Image Anal 2001; 5: 281-299.
[76] Ragnemalm I. The Euclidean distance transform in arbitrary dimensions. Pattern Recognition Letters 1993; 14: 883-888.
[77] Masutani Y, Yoshida H, MacEneaney P, Dachman A. Automated segmentation of colonic walls for computerized detection of polyps in CT colonography. Journal of Computer Assisted Tomography 2001; 25: 629-638.
[78] Nappi J, Dachman A H, MacEneaney P, Yoshida H. Automated knowledge-guided segmentation of colonic walls for computerized detection of polyps in CT colonography. J Comput Assist Tomogr 2002; 26: 493-504.
[79] Hoffman A J, Kruskal J B. Integral boundary points of convex polyhedra. In Linear inequalities and related systems. In: Kuhn H W, Tucker A W, eds. Ann. Math. Studies. Princeton: Princeton University Press, 1956; 223-246.
[80] Fletcher J G, Johnson C D, MacCarty R L, Welch T J, Reed J E, Hara A K. CT colonography: potential pitfalls and problem-solving techniques. Am J Roentgenol 1999; 172: 1271-1278.
[81] Borgefors G. On digital distance transforms in three dimensions. Computer Vision and Image Understanding 1996; 64: 368-376.
[82] Reeders J W A J, Rosenbusch G. Clinical Radiology and Endoscopy of the Colon: Thieme Medical Publishers, 1994.
   The entire contents of each related patent listed above and each reference listed in the LIST OF REFERENCES, are incorporated herein by reference.

### DISCUSSION OF THE BACKGROUND

Colon cancer is the second leading cause of cancer deaths in the United States [41,42,63]. Colonic polyps are seen as potential precursors of colon cancer, and studies show that early detection and removal of the polyps can reduce the risk and the incidence of colon cancer [1,2,43]. Virtual endoscopy of the colon, often called computed tomographic colonography (CTC) or virtual colonoscopy, is a new technique for non-invasive detection of colorectal neoplasms by use of a CT scan of the colon [2,3,4,64]. If CTC can demonstrate satisfactory performance in the detection of polyps, it could be an effective technique for screening of colon cancer in large populations [4,5,65].

To be a clinically practical tool for screening of colon cancer, CTC must be feasible for the interpretation of a large number of images in a time-effective fashion [6,7], and it must facilitate the detection of polyps with high accuracy [4]. However, at present, the interpretation of CTC is a potentially slow process. Despite recent advances in the development of 3-D imaging software and hardware, the current interpretation time of CTC is at least 5-20 minutes per case even for expert abdominal radiologists, and human factors such as fatigue may lengthen the interpretation time, particularly when a primary 2-D interpretation technique based on multiplanar reformatting is used [2]. Moreover, the reported diagnostic performance of CTC varies substantially among readers and among different studies. Although several studies have yielded by-polyp detection sensitivities of 70-90% with corresponding by-patient specificities of approximately 90-100% [8,44], some studies have reported much lower performance results [9].

The accuracy of the detection of polyps is often affected by image display methods because they change the visibility and conspicuity of the polyps. Therefore, a suboptimal implementation of the display methods may increase the perceptual error even among experienced readers [10]. To facilitate a rapid and accurate interpretation of CTC, a number of computer-assisted visualization methods have been reported during the past several years [2,11]. Most of them are concerned with the visualization of the entire colon, so that observers do not miss any lesions. Volume-rendered 3-D endoluminal viewing is a widely accepted method for simulating the appearance of optical colonoscopy [12], and multiple windows may be used for presenting a full 360-degree view of a colonic lumen at a given location [11]. The colon may be unfolded to represent it as a 2-D plane [13,14], which could potentially minimize the blind spots that are often associated with an endoluminal fly-through view [15]. Another method, based on volume rendering, is a semi-transparent view of the complete colon, similar to a double-contrast enema, that can be used for displaying the locations of the findings for a follow-up examination [16].

In these visualization methods, the standard practice has been to color the colon by use of a monochromatic coloring scheme, such as a grey-scale or pink-scale coloring based on the CT values within the data set. A careful, and therefore time-consuming, interpretation of the CTC data is required, because the visual color cues that normally exist in colonoscopy, such as mucosal color changes, are absent. For example, as shown in FIG. 1, diverticula may look quite similar to polyps in a volume-rendered endoluminal grey-scale view. This may lengthen the interpretation time of CTC, because a radiologist may need to study the locations of diverticula to differentiate them from polyps, either by use of 3-D navigation or by adjustment of the shading and lighting effects.

Pseudo-coloring (or indexed coloring) is a well-known method for enhancing the visualization of data that do not have inherent color information [17,18]. It can be used, for example, to highlight important values or to identify structures, features, or patterns within the data. The input data are typically colored by substituting each data value by a color indicated by a lookup table. Also in CTC, some previously reported investigations have used such color schemes for the visualization of colonic lesions. For example, Choi et al. [19] used a color spectral imaging scheme to enhance the visualization of axial CTC images. Summers et al. [20] visualized the results of a computerized polyp detection scheme by coloring of the colon based on the curvature of the colonic surface. Wax et al. [21] developed a translucent volume rendering method for the analysis of mucosal opacities, which allowed one to differentiate between contrast-enhanced stool and polyps. Others have developed color schemes for the visualization of the texture differences between polyps and stool [22,23].

Because the diagnosis of a CTC case is made by radiologists based on visual examination, an unambiguous display of the colon is expected to improve the reader performance in CTC.

Moreover, although recent advances in virtual endoscopy allow physicians to examine the colon in CTC interactively with endoluminal perspective views [66], complete navigation through the entire colon with virtual endoscopy can be tedious and time consuming because an inexperienced physician may lose track of the position and orientation of the colon. The difficulty and the time required for navigating through the colon could be reduced substantially by use of the centerline of the colonic lumen. Directing a 3-D camera that creates the endoluminal views of the colon along the centerline allows physicians to navigate easily through the colon. A physician can also veer from the path for closer inspection of the anatomy and later use the central path to re-orient him-or herself in the virtual space. The colon centerline can also aid an advanced visualization of the colon, such as straightening the colon [13,67] and the cylindrical and planar-map projections of the colon [68].

Several approaches have been proposed for computation of the centerline of a colon, some of which are semi-automatic, whereas others are fully automatic. Ge et al. [69, 70] used topological thinning for creating a 3-D skeleton, then removed loops and branches and, finally, approximated the centerline by using B-splines. A user defines a starting seed point. Paik et al. [71] generated an initial path at the object surface by using region-growing. The path was later refined to a center position by use of a thinning algorithm. The user supplied the program with two end points. Samara et al. [72] used a seed point in the rectum as the start for a region-growing that generated a centerline. The same procedure was repeated for another seed point placed in the cecum. The final centerline was the average of the two centerlines. Wan et al. [73] used a distance from boundary field to create a directed, weighted graph. The centerline was calculated by computation of the minimum-cost spanning tree for the graph. One or two pre-defined seed points were needed as input. Bitter et al. [74] computed the gradient field of a distance from boundary field, which was first used for computing the distance from the source and the root in order to find the end points, and then to find the actual centerline. This centerline could later be expanded to a skeleton based on the same basic principle. The method required a single connected component as input, but no seed points were necessary. Deschamps and Cohen [75] used one or two user-defined seed points as a starting (ending) point for a front propagation that was used for finding an initial distance map. A second, inwardly propagated front was then computed, and a minimal path search for this map was performed which provided the final centerline.

Most of these existing methods rely on manually extracted seed points or complete colon segments identification for the computation of a single centerline segment. Manual extraction of the seed points can be a tedious and inaccurate process when the location of the colon is obscured by a large amount of small bowel adhering to the colon, or when the colon is fragmented into multiple disconnected segments due to collapsed regions. For the same reasons, the segmentation of the colon is often incomplete, and it contains extra-colonic segments in addition to the colon. This makes methods that rely on a complete colonic pathway and complete colon segments identification work poorly unless some additional seed selection procedure is applied for finding the different parts of the colon. Fully automated methods can be used even in cases with collapsed regions by iterating through each independently segmented region. However, in this way, centerline segments will also be created for each extra-colonic findings.

The existing algorithms for the computation of a colon centerline are also time-consuming. At best, a computation time of 12-17 seconds per centerline, evaluated on 4 cases with use of an Intel Pentium-based 700 MHz PC, was reported [73]. This computation time excludes the time for the Euclidean distance transform (DT) [76] needed by the algorithm. Because the Euclidean DT calculation itself is a time-consuming process, the actual centerline computation time is considerably longer. Other approaches require even more computation time, such as 5 minutes (Silicon Graphics O₂ workstation) [72] and 8 minutes (Silicon Graphics R10000 workstation) [69].

Finally, problems such as the potentially slow interpretation time and the variable diagnostic performance of CTC may also be addressed by use of computer-aided detection (CAD).

CAD could complement CTC by reducing the interpretation time, minimizing the perceptual error, and increasing the diagnostic performance of the interpretation of CTC [2]. Several CAD schemes have been developed for the detection of polyps in CTC during recent years. Most use shape-based features to locate suspicious regions from the colonic wall: Vining et al. [45] used curvature and wall thickness features of the colonic surface, Summers et al. [20,46] used curvature and sphericity features, Paik et al. [47,48] used the Hough transform, Yoshida et al. [33,49] used shape and texture features with a fully automated scheme, and Kiss et al. [50] used surface normals and sphere fitting. In addition, several methods have been developed for the reduction of false-positive (FP) detections. Göktürk et al. [51] developed a random orthogonal shape sections method for the differentiation of polyp candidates by use of support vector machines, Näppi et al.[52] developed several volumetric shape and texture methods for the differentiation of the shape and the texture of polyp candidates, Acar et al. [53] used edge displacement fields to model the changes in consecutive cross-sectional views of the CTC data, Jerebko et al.[54] used a committee of neural networks to classify polyp candidates, and Näppi et al.[35] developed a new method for the extraction and analysis of polyp candidates. The evaluation methods and the results vary among studies; generally, however, the results indicate that 70-100% of visible polyps ≧10 mm could be detected by CAD with an average FP rate of 1-8 detections per data set.

Colorectal masses are generally considered well visualized by a radiologist because of their size, continuation, and secondary findings. Therefore, little effort has been made to develop CAD schemes for mass detection. Nevertheless, the detection of both polyps and masses by CAD in CTC would provide a more comprehensive computer aid than the mere detection of polyps. If masses are not detected by CAD, the radiologist needs to perform a careful and complete review of all CTC cases for the presence of masses, which may increase the reading time. In addition, not all masses are easy to detect, depending on the readers' experience and on how rapidly the readers are reading the cases [55]. Therefore, the application of CAD for mass detection could improve the diagnostic accuracy of CTC by reducing potential reading errors due to reader fatigue, inexperience, or too rapid reading. Without explicit mass detection, the computer could also confuse masses with other types of lesions. Some types of masses have local surface bumps that tend to be detected as suspicious regions by a CAD scheme for the detection of polyps (FIG. 16A). Because of the large size of masses as compared with polyps, the mass surface could be detected as several such FP polyp detections, thereby potentially confusing the radiologist when the case is reviewed with CAD.

Previously, a multi-scale method for the detection of masses in CTC was developed [56]. Cap-like shapes were first detected by use of the shape index and the curvedness features in multiple scales based on a Gaussian pyramid of the CTC volume. The mass regions were extracted by use of a region-growing technique, and false positives were reduced by a quadratic classifier. In an evaluation with 20 patients, including 7 patients with a total of 8 colonoscopy-confirmed masses, all masses were detected by the method with an average FP rate of 0.45 detections per patient. Although the detection performance was satisfactory for that preliminary study, the method was well suited only for the detection of masses with a significant intraluminal component. Furthermore, the method added considerable computational overhead to the CAD scheme because it was implemented separately from the polyp detection scheme.

### SUMMARY OF THE INVENTION

The present invention provides a new method for enhancing the endoscopic visualization of the colonic lumen. Each unique structure of importance in the colon can be highlighted automatically by a unique color, which makes it easy to perceive the relevant lesions at one glance. The present invention provides a method for coloring of the colon, which is based on a detailed theoretical framework of shape-scale analysis and can be adjusted systematically to delineate specific types of lesions. After the shape-scale specification, the different colonic structures are marked automatically with a unique color to provide an immediate visual differentiation of the lesions. In particular, polyps and diverticula, the visual differentiation of which may require additional time-consuming steps in monochromatically volume-rendered images (FIG. 1), can be differentiated with little effort by use of our method because they are assigned different colors. Moreover, the input CTC data can be acquired in a conventional manner: there is no need for special protocols such as the use of a contrast agent or fecal-tagging agents. The computations required by the method can also be implemented efficiently. Furthermore, the method can be used in applications of virtual endoscopy other than mere visualization, such as computer-aided detection (CAD), in which the detection of lesions of interest is based upon their shape or scale characteristics. Such an application of our coloring scheme based on shape-scale signatures is described below.

The present invention also provides an algorithm for computation of the colon centerline that is not only robust to collapsed regions and poor segmentation, but is also faster than any previously reported method. The input to the algorithm is a distance map (DM) that is the result of a DT of the segmented colonic lumen. The colonic lumen is segmented by first thresholding of the air in the CTC data set, and then removing the lungs, bones, and skin. The regions representing lungs, bones, and skin are obtained by thresholding, where threshold values are determined adaptively from the histogram of the isotropic volume by identification of the characteristic peaks corresponding to air, fat, and muscle [77]. More precise segmentation algorithms, with respect to complete colon segments identification, exist [78]. However, in order to emphasize on the advantages of the present algorithm, the straightforward threshold-based segmentation approach was chosen.

The centerline algorithm is based on the well-known Kruskal algorithm [79] for generating a minimum spanning tree. In the present invention, the cost function is based on the DM value for the nodes and the Euclidean distance between nodes. Unlike the Kruskal algorithm, the iteration is allowed to stop before a single spanning tree was formed.

The centerline algorithm first extracts local maxima in the DM, divided into two categories—seed points and regular points. Seed points are the subset of local maxima that has DM values larger than a pre-defined threshold level. Regular points are the remaining local maxima. Starting with maxima with the largest distance values, available maxima are iteratively transferred to be single-node graphs in a graph structure. During each iteration, graphs are joined by linking of all pairs of nodes that satisfy a set of connection criteria. After the last iteration, branches are removed from the remaining graphs. The end segments of the remaining graphs are then recovered, after which the actual centerline is represented as the set of graphs containing at least one seed point each.

Further, the present invention provides a CAD method for the detection of intraluminal and non-intraluminal types of colorectal masses. Intraluminal masses are masses with a significant intraluminal component, such as lobulated, polypoid, or circumferential types of masses. Non-intraluminal masses, are masses that are associated with a mucosal wall-thickening type of growth pattern rather than those with a significant intraluminal component. Two mass detection methods were developed, one for each type of mass, because these two types can appear in very different forms in CTC (FIGS. 16A-16C). The first method detects intraluminal masses by an analysis of the fuzzy memberships between the initial detections of suspicious polypoid regions by previously developed CAD methods for the detection of polyps [34]. The second method detects non-intraluminal masses by searching for thickened regions of the colonic wall. The final regions of the detected masses are extracted by use of a level set method based on a fast marching algorithm. To investigate whether the mass detection methods and the polyp detection methods could be integrated into a single CAD scheme, the polyp candidate detections which overlapped with the regions of the final mass detections were eliminated. Thus, each detected lesion was labeled either as a polyp or as a mass.

According to one aspect of the present invention, there is provided a method, system and computer program product for displaying volumetric image data of a target organ as a visual aid in detecting at least one abnormality in the target organ, comprising (1) obtaining the volumetric image data of the target organ, the volumetric image data including a plurality of voxels having a respective plurality of image data values; (2) determining at least two geometric feature values at each voxel in the plurality of voxels based on the plurality of image data values; (3) determining a colormap by assigning a color to each possible combination of the at least two geometric feature values, wherein each assigned color corresponds to a different region type within the target organ; (4) determining a display color for each voxel in the plurality of voxels based on the determined colormap; and (5) displaying, based on the determined display color for each voxel in the plurality of voxels, a color image representing the volumetric image data of the target organ.

Further, according to one aspect of the present invention, there is provided a system for displaying volumetric image data of a target organ as a visual aid in detecting at least one abnormality in the target organ, comprising: an image acquisition unit configured to obtain the volumetric image data of the target organ, the volumetric image data including a plurality of voxels having a respective plurality of image data values; a processor configured (1) to determine at least two geometric feature values at each voxel in the plurality of voxels based on the plurality of image data values, (2) to determine a colormap by assigning a color to each possible combination of the at least two geometric feature values, wherein each assigned color corresponds to a different region type within the target organ, and (3) to determine a display color for each voxel in the plurality of voxels based on the determined colormap; and a display unit configured to display a color image representing the volumetric image data of the target organ, based on the determined display color for each voxel in the plurality of voxels.

Further, according to another aspect of the present invention, the step of determining the colormap comprises obtaining simulated or clinical volume data representing the target organ; determining, based on the simulated volume data, the at least two geometric feature values for each voxel in the simulated volume data; selecting a region type of the target organ; determining combinations of the at least two geometric feature values that most frequently correspond to the selected region type; and assigning a color to each of the determined combinations of the at least two geometric feature values that correspond to the selected region type.

Further, according to another aspect of the present invention, the step of determining the at least two geometric features comprises: determining, based on the plurality of image data values, a shape index at each voxel, the shape index being indicative of a local shape at each respective voxel; and determining, based on the plurality of image data values, a curvedness index at each voxel, the curvedness index being indicative of a magnitude of the local shape at each respective voxel.

Further, the present invention provides a method of processing a set of volumetric data encompassing a target organ, comprising detecting multiple lobulated regions of the targeted lesion; and reclassifying the detected regions to detect the entire lesion. In addition, the present invention provides that the detecting step comprises computing volumetric features characteristic of lobulated regions at each point in the volume representing the targeted lesion; and determining the possible lobulated regions of the targeted lesion based on the computed volumetric features. Further, the reclassifying step comprises merging the detected lobulated regions by fuzzy merging; and expanding the merged lobulated regions to extract the entire lesion. Moreover, the merging step comprises determining the membership grade for each of the detected lobulated regions; and clustering the lobulated regions that have a high membership grade. The expanding step comprises applying a volumetric region expansion method to the merged lobulated regions, and the step for determining the membership grade comprises formulating a membership function for any two lobulated regions, which has a high value when the two regions belong to a single lesion, based on the CT and gradient values along a ray that connects the two regions; and calculating a membership grade for each of the lobulated regions based on the membership function. Finally, the step for applying a volumetric region expansion comprises using a modified level set method with fast marching algorithm.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a volume-rendered endoscopic view of diverticula (indicated by four white arrows), wherein the diverticula look very similar to small polyps when the standard grey-scale coloring scheme is used for visualizing the colonic lumen such that radiologists need to spend time to determine whether these lesions are diverticula or polyps, which increases the interpretation time of CTC;

FIG. 2 illustrates that the shape index feature maps local surface patches continuously to a numeric value between −1.0 and 1.0;

FIG. 3 is an illustration of the shape-scale spectrum, wherein the 15 surface patches shown in the plane demonstrate the shape and scale information provided by the local shape index and curvedness values at each region;

FIGS. 4A and 4B show examples of computer-detected lesions, including a detected polyp (4A) and four detected diverticula (4B);

FIGS. 5A-5D show examples of computer-simulated lesions, including folds (5A), sessile polyps (5B), pedunculated polyps (5C), and diverticula (5D);

FIGS. 6A and 6B are illustrations of the shape-scale spectra of computer-simulated lesions (6A) and a clinical data set (6B), wherein the clinical data set contains 5 colonoscopy-confirmed polyps and a large number of diverticula and the white color indicates highest and black color zero occurrence of a shape index and curvedness pair;

FIG. 7 is a diagram of the shape-scale color map method according to the present invention;

FIG. 8 is an example view of a CAD workstation for the detection of polyps and masses in CTC;

FIGS. 9A-9I show examples of the shape-scale color maps, including color maps generated by use of computer-simulated lesions (9A-9F), color maps generated by use of clinical data (9G-9H), and a color map generated for application of the hybrid scheme (9I);

FIGS. 10A-10D are examples of the coloring of computer-simulated lesions, including folds (10A), sessile polyps (10B), pedunculated polyps (10C), and diverticula (10D);

FIGS. 11A-11D show an endoscopic view of a polyp (11A) and three examples (11B-11D) of colorings of the view of 11A by shape-scale color maps based on computer-simulated lesions;

FIGS. 12A-12D show an endoscopic view of a polyp and diverticula (11A) and three examples (12B-12D) of colorings of the view of 12A by shape-scale color maps based on clinical data;

FIGS. 13A and 13B illustrate two examples of a monochromatic endoscopic view of a polyp (left) and the corresponding hybrid shape-scale coloring (right) where the computer-detected polyps and diverticula are colored in green and blue, respectively;

FIG. 14 illustrates ROC curves, wherein the thick ROC curves represent the performance of observers 1, 2, and 3 without the use of shape-scale coloring, the thin curves represent the performance with the application of the hybrid shape-scale color map, and the three thin curves overlap each other significantly and thus appear to be almost a single line.

FIG. 15 illustrates the effect of the hybrid shape-scale color map in improvement of the observer performance, wherein the black bar shows the performance with monochromatic coloring of the colon, and the grey bar shows the performance when the colon was colored by the hybrid coloring scheme;

FIGS. 16A-16C illustrate examples of masses with different types of morphologies, including, respectively, (a) a 50-mm intraluminal circumferential mass with apple-core morphology in which the irregular surface of the mass produces locally polyp-like shapes that are detected as polyp candidates (white regions) by a CAD polyp detector; (b) a 35-mm polypoid pedunculated mass (white arrow); (c) a non-intraluminal wall-thickening type of mass, which can be identified based on the associated mucosal thickening, wherein the white arrows indicate the margins of the mass, and the mass diameter perpendicular to the colonic pathway is approximately 35 mm, but the complete length of the colonic segment covered by the thickened wall is approximately 90 mm;

FIG. 17 illustrates a distribution of the diameters of the 14 masses used in the mass study;

FIG. 18 illustrates how the region of the colonic wall is extracted by use of a knowledge-guided colon segmentation technique in which the top figures show that, starting from a 3-D isotropic data set, the technique first eliminates the region outside the body, the osseous structures, and the lung bases, and the bottom figures show that the colonic wall is extracted from the remaining region first by use of thresholding (left), and then by use of a self-adjusting volume-growing technique (right), wherein the volume-growing step eliminates the small bowel and other extra-colonic structures from the extracted region of the colonic wall (in the axial images at the bottom, the extracted region of the colonic wall is shown in white before and after the volume-growing step);

FIGS. 19A and 19B illustrate the method in which (a) to estimate whether two detections, A and B (indicated by thick white boundaries), are within the same lesion, a sampling ray R is determined between the centers of A and B, and to characterize the membership between A and B, R was divided into two regions: [A, B] is the region of R within A and B, and (A, B) is the region of R outside A and B; and (b) the ratio of the smallest CT value along R outside A and B (min_{CT} (A, B)) to the average CT value of A and B along R (mean_{CT}[A, B]) is determined wherein a high ratio of

$$\frac{{min}_{CT} ( A , B )}{{mean}_{CT} [ A , B ]}$$

indicates that A and B are located within the same lesion;

FIGS. 20A and 20B show dotted lines that represent a sampling ray between a polyp detection and a mass detection in the axial (20A) and coronal (20B) view, wherein the membership grade of the detections based only on the CT value and gradient would be 0.56 and because the detections are far apart (3 cm), they are not merged because the distance term ε_{D}(A,B) reduces the membership grade below the hard threshold of 0.5;

FIG. 20C shows an example of the merging of detections on a non-trivial shape, wherein the circles A, B, and C represent initial detections and even though A and B have a membership grade of zero because of the air in between, they are merged into the same region because C has a high membership grade with both A and B, and the distance between A, B, and C is small;

FIGS. 21A and 21B show, respectively, that the thickness of the colonic wall is estimated by use of a 3-D sampling ray between L_{begin} and L_{end} and after determining the location of the maximum CT value, CTₘₐₓ, along the ray, the region of the colonic wall is determined from the locations of the maximum gradients, GR_{begin} and GR_{end}, around CTₘₐₓ; and that the symmetry of the thickening is verified by projection of an inverse sampling ray from the starting point L_{begin}, to the opposite wall, and searching for detected thickening in the neighborhood of the “checkpoint” on the wall;

FIG. 22A shows residual fluid (indicated by the white boundary) may look similar to a thickened colonic wall, because it appears as a clearly delineated layer of high-density CT values at the surface of the colonic wall, wherein such a region can be detected based on its flat surface where the gradient vectors are oriented in the sagittal direction;

FIG. 22B shows that a normalized vector \overline{v} at a surface of fluid is expected to have a sagittal component of ≧90% of the magnitude of the vector;

FIGS. 23A-23C show three examples of the detection and level-set extraction of masses in axial and coronal images, wherein the first and second rows show the original mass regions in soft tissue and lung window settings, respectively, and the third and fourth rows show the detected and extracted regions of the masses, respectively, labeled in white, wherein FIGS. 23A and 23B show detection of intraluminal masses by use of the fuzzy merging technique and FIG. 23C shows detection of a non-intraluminal mass by use of a wall-thickening analysis technique;

FIG. 24A shows a distribution of the TP (large black circles) and FP (small grey circles) mass detections, wherein the boundaries indicate various detection sensitivity levels generated by a quadratic classifier, such that FIG. 24A shows detection of intraluminal masses by the fuzzy merging method, wherein the discriminating features are the mean CT value (mean(CT)) and the mean of the 10 highest shape index values within the mass (max10(SI)) in that these features are effective because masses have a relatively uniform CT value that is generally higher than that of FPs, and their shape has more of the smooth cap-like surface patches than does that of FPs;

FIG. 24B shows detection of non-intraluminal masses by wall-thickening analysis, wherein the discriminating features are the variance of the CT value(var(CT)) and the mean value of the gradient (mean(GRM)) in that these features characterize the sharpness of the surface of the non-intraluminal masses: FPs tend to have a more blurred surface because they are often associated with nonspecific stool or collapsed regions of the colon;

FIG. 25 illustrates the FROC curve (solid line) of the combined performance of the detection of 14 masses (30-60 mm) from the 82 patients with the fuzzy merging and wall-thickening analysis in which the performance of the detection of 30 polyps (5-25 mm) by the CAD scheme for the detection of polyps is shown by the dotted curve;

FIG. 26 illustrates visualization of the mass (arrows) that was missed by both mass detection methods in which the rectal mass was partially cut off from the CTC data set in both supine and prone positions, including: top left: coronal view shows the tip of the mass projecting into the air-filled rectum within caudal CT images; right: the most caudal supine axial CT image suggests the presence of a mass; and bottom left: 3-D endoscopic view confirms the characteristic apple-core morphology of the mass;

FIGS. 27A and 27B illustrate examples of FP mass detections, indicated by white arrows including, respectively, axial (top) and coronal (bottom) views (27A) of a bulbous ileocecal valve detected by the fuzzy merging method; and soft-tissue window displays of the axial (top) and coronal (bottom) views (27B) of a normal circumferential thickening due to perirectal muscles and wall, detected by the wall-thickening analysis, wherein the rectal tube is in place with the balloon deflated. All FPs detected by the wall-thickening analysis were of this type;

FIG. 28 shows an example of an intraluminal mass that was not detected by the CAD method in the supine position, wherein the white arrows indicate the location of the mass in the axial, coronal, and sagittal images and the mass is immersed within fluid in the supine position (top row), but is clearly visible in the prone position (bottom row);

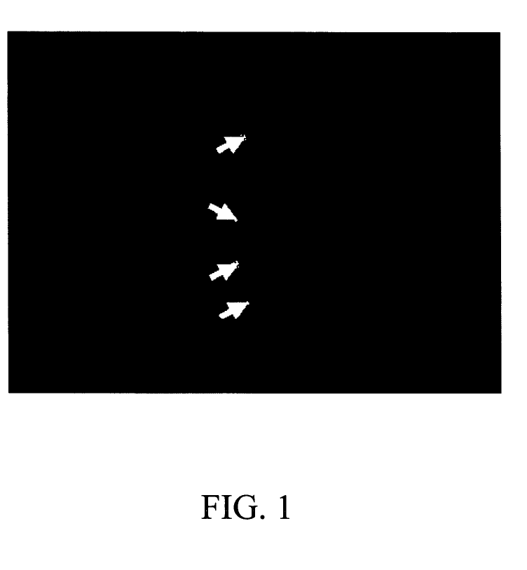

FIGS. 29A-29G shows a step-by-step illustration of the principle of the centerline algorithm, although each figure is not an actual rendering by use of the algorithm, but is a simplified illustration in order to make the principle as clear as possible, wherein the actual algorithm works in 3-D, using a 3-D distance map and spheres rather than a 2-D distance map and circles, the steps including:
29A, the Seedpoint Extraction step, in which an initial set of maxima, referred to as the seed points, is chosen from the set of available maxima found in the Detection of Local Maxima in DM, wherein the circles surrounding the seed points represent the scaled central distance values;
29B, in which links joining pairs of nodes satisfying the criteria in the Graph Connection are created;
29C, in which, as a part of the Window-Based Iteration, more maxima found in the Detection of Local Maxima in DM are chosen;
29D, in which the graph generated after the Graph Connection step in Window-Based Iteration is applied;
29E, in which links removed in the first step of Branch Cutting are shown as dashed lines;
29F, in which all links removed in the first and second steps of BranchCutting are shown as dashed lines and because no nodes have more than two links, the branch-cutting process stops;
29G, in which, in Branch Recovery, the branches of the current end nodes B and C are recovered and because at least one of the nodes in the graph is a seed point (B, among others), the solid links represent a centerline segment that is stored in Save Result;

FIG. 30 illustrates a method for measurement of the similarity between a “reference” centerline (solid line) and a “slave” centerline under evaluation (dashed line), including calculating the percentage of the regions of the reference centerline that are covered by the slave centerline, wherein the segments and above illustrate regions not covered; calculating the average and maximum displacements (in 3-D) between the reference and slave centerlines in the regions covered by the slave centerline, which is performed by averaging of the displacements between the reference and slave centerlines, measured at an equal path distance at the reference centerline, where applicable;

FIGS. 31A-31I show representative cases in the “straightforward” category (31A-31C), representative cases in the “moderate” category (31D-31F), and representative cases in the “challenging” category (31G-31I);

FIGS. 32A-32D illustrate, respectively, a phantom with small average radius (3 mm) (32A), a phantom with large average radius (18 mm) (32B), a centerline computed for the most narrow phantoms (2 mm, 3 mm radius) rendered as grey, compared with path used for generating the phantom (white) (32C), and a centerline computed for the least narrow phantom (21 mm radius) rendered as grey, compared with path used for generating the phantom (white) (32D), wherein displacement is large for some areas because the phantom is so wide, and thus the natural central path has changed;

FIGS. 33A and 33b show a perspective rendering of the colonic lumen with computer-generated centerline (33A); and a perspective rendering of the displacement at each location in 3-D between the human-and computer generated centerlines (33B);

FIG. 34 illustrates the steps in a method of displaying volumetric image data of a target organ using a colormap according to the present invention; and

FIG. 35 illustrates a system for displaying volumetric image data of a target organ using a colormap according to the present invention.

### DETAILED DESCRIPTION OF THE PREFERRED EMBODIMENTS

The description of the present invention includes a written description of: (1) visualization of the colon by shape-scale signatures; (2) computation of the colon centerline in CT colonography; and (3) detection of colorectal masses in CT colonography based on fuzzy merging and wall-thickening analysis.

I. Visualization of the Colon by Shape-Scale Signatures

Local Shape Index and Curvedness

The local shape index and curvedness measure the shape characteristics of a local surface patch at a point, and they form the basis for defining the shape-scale spectrum which is described in the next section. Both quantities are defined based on the notion of the principal curvature. A traditional approach for computing the principal curvatures of 3-D data is to fit a parametric surface to the data and compute its differential characteristics in a local coordinate system [24]. However, parameterization of surfaces with a complex topology is complicated, and curvature information is often needed before these surfaces are generated. Therefore, the differential characteristics of the iso-surfaces were computed directly from the 3-D data volume, without extraction of any surfaces, based on an implicit representation of the iso-surface [25,26]. That is, the differential characteristics are computed locally at each voxel by use of the differentials of the volume in three directions. The interpretation is that the computed differential characteristics at voxel p correspond to the differential characteristics of the iso-surface passing through p. The definition and the computational method of the local shape index and the curvedness are described briefly in the following.

Let h(p) denote a voxel value (typically, the CT value) at a voxel p=(x,y,z)∈IR³. A 3-D iso-surface P at a level a is given by
P={p=(x,y,z)∈IR³;h(p)=a}.
To obtain an infinitely differentiable 3-D function, the volume is convolved with a Gaussian filter. The implicit-function theorem indicates that, at each voxel p, there exists a small neighborhood U of p in which z can be expressed by a smooth function φ of x and y. By denoting (x, y) with (u, v) in this neighborhood, the iso-surface P can be represented in a parametric 2-D form as
P={(u,v)∈IR²;h(u,v,φ(u,v))=a}.
The principal curvatures are obtained as the eigenvalues of the following matrix [27], called the Weingarten endomorphism:

$$\begin{matrix}W = {( \begin{matrix}E & F ( 3 ) \\ F & G\end{matrix} )}^{- 1} ( \begin{matrix}L & M ( 4 ) \\ M & N\end{matrix} ) \\ = \frac{1}{EG - {F}^{2}} ( \begin{matrix}GL - FM & GM - FN \\ EM - FL & EN - FM\end{matrix} ) .\end{matrix} \qquad ( 1 )$$

Here, E, F, and G are the first fundamental forms [27] defined at a voxel p as
E≡Pᵤ·Pᵤ, F≡Pᵤ·Pᵥ, G≡Pᵥ·Pᵥ, (2)
where Pᵤ and Pᵥ are the partial derivatives of P in terms of u and v at p, respectively. In the following, a suffix of u, v, x, y, or z indicates the partial derivative in terms of these variables, and all of the quantities are defined on a single voxel p unless mentioned otherwise. L, M, and N are the second fundamental forms [27] defined as
L≡Pᵤᵤ·Q, M≡Pᵤᵥ·Q, N≡Pᵥᵥ·Q, (3)
where Q is a normal vector to the iso-surface P defined by Q=Pᵤ\hat{ }Pᵥ.
To calculate the principal curvatures, the implicit differentiation and the chain rule is used to obtain Pᵤ=∂P/∂u=(1,0,∂φ/∂u)=(1,0,hₓ/h_{z}). Substituting similar expressions for Pᵥ, Pᵤᵤ, Pᵤᵥ, and Pᵥᵥ in Eqs. (3)-(7), one can compute the determinant of the matrix W, called the Gaussian curvature, K, and half of the trace of W, called the mean curvature, H, as follows:

$$K ≡ det ( W ) = \frac{LN - {M}^{2}}{EG - {F}^{2}} = \frac{1}{\vert h {\vert}^{2}} {\sum }_{( i , j , k ) = Perm ( x , y , z )} \{ {h}_{i}^{2} ( {h}_{jj} {h}_{kk} - {h}_{jk}^{2} ) + 2 {h}_{j} {h}_{k} ( {h}_{ik} {h}_{ij} - {h}_{ii} {h}_{jk} ) \} , \\ H ≡ \frac{1}{2} trace ( W ) = \frac{EN - 2 FM + GL}{2 ( EG - {F}^{2} )} = \frac{1}{\vert h {\vert}^{3 / 2}} {\sum }_{( i , j , k ) = Perm ( x , y , z )} \{ - {h}_{i}^{2} ( {h}_{jj} + {h}_{kk} ) + 2 {h}_{j} {h}_{k} {h}_{jk} \} .$$

Here, Perm(x, y, z) denotes a permutation of (x, y, z), i.e., Perm(x,y,z)≡{(x,y,z),(y,z,x),(z,x,y)}. Because the principal curvatures κ₁ and κ₂ are the eigenvalues of the Weingarten endomorphism of Eq. (3), the Gaussian curvature and the mean curvature can also be defined by [28]

$$K = {κ}_{1} {κ}_{2} , H = \frac{{κ}_{1} + {κ}_{2}}{2} . \qquad ( 4 )$$

From Eq. (4), it is easy to derive that the principal curvatures at the voxel p can be expressed as
κᵢ(p)=H(p)±\sqrt{H²(p)−K(p)}, i=1,2.
Let us denote κₘᵢₙ=min{κᵢ,κ₂} and κₘₐₓ=max{κ₁,κ₂}. Then the local shape index S(p) and the local curvedness C(p) at the voxel p are defined as [29]

$$\begin{matrix}S ( p ) = - \frac{2}{π} arctan \frac{{κ}_{max} ( p ) + {κ}_{min} ( p )}{{κ}_{max} ( p ) - {κ}_{min} ( p )} , & ( 5 ) \\ C ( p ) = \frac{2}{π} ln \sqrt{( {{κ}_{min} ( p )}^{2} + {{κ}_{max} ( p )}^{2} ) / 2.} & ( 6 )\end{matrix}$$

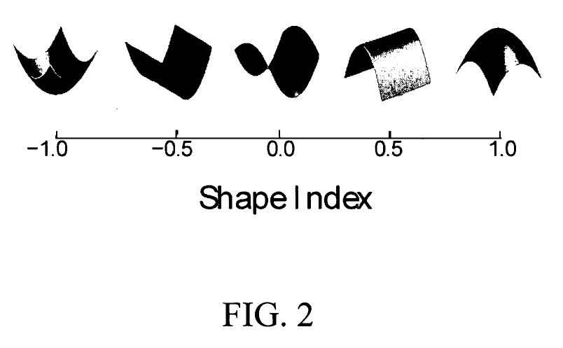

The local shape index measures the shape of a local iso-surface patch at a voxel. Every distinct shape corresponds to a unique value of the shape index with a value range of [−1,1] (FIG. 2). Because the transition from one shape to another occurs continuously, the shape index can describe subtle shape variations effectively. The local curvedness is a measure of the size or scale of the shape and represents how gently curved the iso-surface is. The dimension of the curvedness is that of the reciprocal of length, and its range is ]−∞,∞[. Curvedness is a “dual” feature of the shape index in that the shape index measures “which” shape the local neighborhood of a voxel has, whereas the curvedness measures “how much” of the shape the neighborhood includes. A small value of curvedness implies a smooth surface patch, whereas a large value implies a very sharp knife-like surface patch.

The idea of determining the local shape and its “scale” at each voxel is somewhat similar to multi-scale methods, where an input signal (e.g., image data) is typically divided into multiple new signals, each representing the original signal at a unique scale [30,31,32]. The original signal may then be studied and manipulated independently at various scales. However, it is sometimes challenging to establish a useful relationship between the various scale representations of the same signal. In contrast, the local shape index and the curvedness capture the intuitive notion of the local shape of an iso-surface at a voxel, and provide an efficient representation of the colonic surface for our application.

The Shape-Scale Spectrum

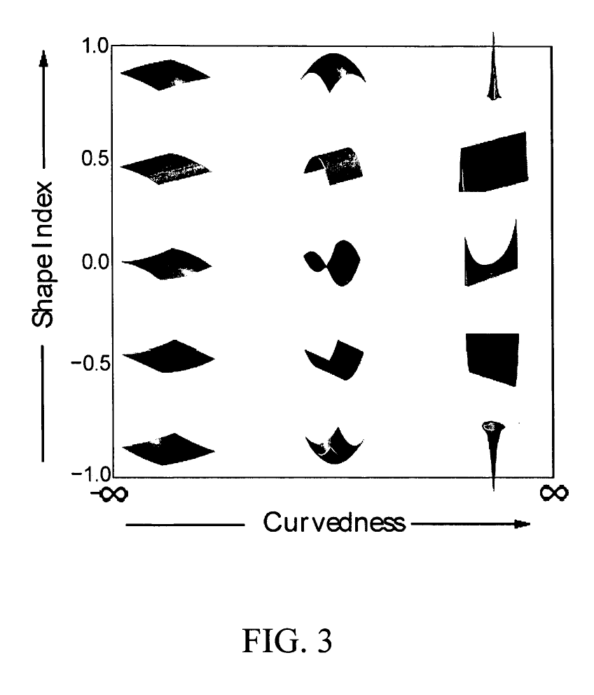

The local shape index and curvedness value pair (S(p), C(p)) of each voxel p of a 3-D data set, defined by Eqs. (5) and (6), can be used for constructing a mapping from the voxels of the data set to a Cartesian plane called the shape-scale spectrum (FIG. 3) [29]. When (S(p), C(p)) are mapped into the shape-scale spectrum, the lesions that can be differentiated from each other based on their shape or scale are represented by unique patterns. Here, one can call such patterns shape-scale signatures. When each shape-scale signature is marked with a unique color, one obtains a 2-D color map that can be used for coloring 3-D data sets for an effective visual differentiation of the lesions of interest.

The differentiation of colonic polyps, diverticula, folds, and the colonic wall was examined. Each of these lesions has some unique characteristics. Polyps are generally bulbous, locally cap-like structures that adhere to the colonic wall or to a fold. Diverticula are small, cup-like structures within the colonic wall. Folds are elongated, ridge-like structures that arise from the colonic wall. The underlying colonic wall can be characterized as a rut-like spinode. The shape index values of these lesions are ideally S≈1 for polyps, S≈−1.0 for diverticula, S≈0.5 for folds, and S≈−0.5 for the colonic wall. The curvature values are expected to be high for polyps and diverticula, relatively low for the colonic wall, and moderately high for folds. The highest values of curvedness do not occur in CTC data because of the lack of sharp biological objects and the smoothing effects of the CTC image formation process.

Generation of Shape-Scale Signatures

There are two methods for generating shape-scale signatures of the colonic lesions of interest. The first method derives the signatures from computer-detected lesions in real clinical data, and the second method derives the signatures from computer-simulated lesions that imitate the shape of the lesions of interest. Real clinical data are expected to produce more realistic shape-scale signatures than do those generated by computer-simulated lesions; however, obtaining relevant clinical data is complicated. On the other hand, the development and evaluation of realistic computer-simulated phantoms can be challenging, but their shape-scale signatures can be obtained accurately because the regions of the simulated lesions are known precisely. If the computer-simulated lesions are sufficiently realistic, they may also be used for testing the effect of a color map on the coloring of colonic structures, and several different examples of a lesion can be simulated with ease by appropriate variation of the simulation parameters.

Use of Clinical Data

The clinical data used in this study were obtained from the CTC examinations performed during 1997-2001 at the University of Chicago Hospitals. Each patient underwent standard pre-colonoscopy cleansing, and the colon was insufflated with room air. The CTC scanning was performed with a helical CT scanner (GE 9800 CTi or LightSpeed QX/i; GE Medical Systems, Milwaukee, Wis.) in the supine and prone positions. The collimation was 2.5-5.0 mm, the pitch was 1.5-1.7, the reconstruction interval was 1.5-2.5 mm, the current was 60-100 mA with 120 kVp, and the matrix size of the axial images was 512×512, with a spatial resolution of 0.5-0.9 mm per pixel. Optical colonoscopy was performed on the same day as the CTC, and radiologists confirmed the presence and location of polyps and diverticula in the CTC data sets by use of colonoscopy reports, pathology reports, and a GE Advantage Windows workstation (GE Medical Systems, Milwaukee, Wis.). Fourteen patients had a total of 21 colonoscopy-confirmed polyps; 20 polyps were 5-12 mm, and one was 25 mm in diameter.

To determine the shape-scale signatures of actual colonic lesions, four CTC data sets which contained pedunculated polyps, sessile polyps, and dozens of diverticula were selected. The polyps were detected by use of our previously developed fully automated CAD scheme for the detection of polyps from CTC data sets, which is described briefly below. Diverticula were detected by use of a new method. Folds were sampled from among false-positive detections as described below.

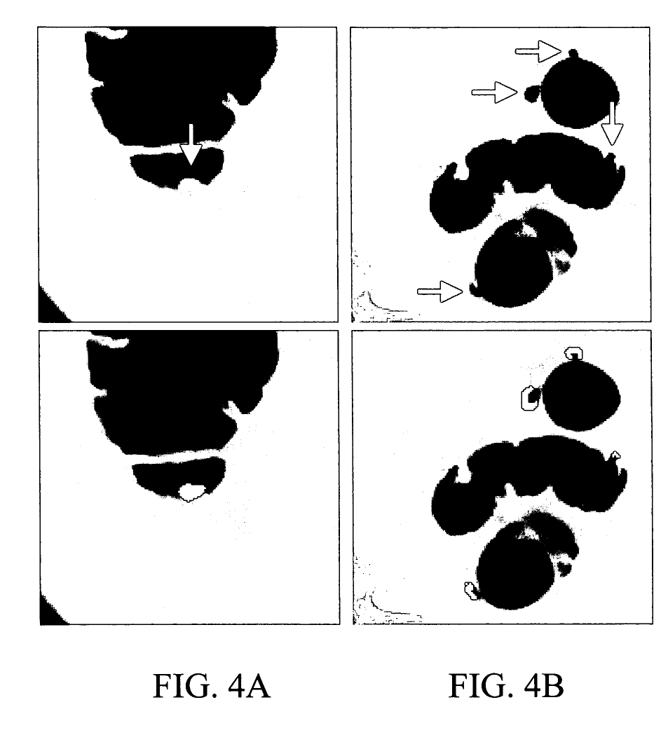

The CAD scheme for the detection of polyps has been described in detail elsewhere [33,34,35]. Briefly, each input data set is interpolated linearly in the axial direction so that isotropic volumes are obtained, and the region of the colonic wall is extracted by use of a knowledge-guided segmentation method [36]. The polyp-like regions are detected based on the shape index and curvature features with hysteresis thresholding, and extracted by use of conditional morphological dilation [35]. The latter extraction step ensures that the shape-scale signatures of the detected polyp candidates are those of their visually perceived regions rather than those within the range of the predetermined shape index and curvature values for initial detection. FIG. 4A shows an example of a computer-detected polyp. In this study, the final step of the CAD scheme, which is the reduction of false positives, was not performed because this would have eliminated most detections of folds. Instead, 10 examples of polyps and 10 examples of folds were manually selected from among the initial set of the computer-detected polyp candidates.

Because the CAD scheme described above was designed to detect only polyps, it was necessary to develop a separate method for the detection of diverticula. Because the cup-like diverticula are opposite in shape to the cap-like polyps, the diverticula can be detected similarly to polyps, but by mirroring the values of the shape index used to search for suspicious regions. That is, whereas polyps are detected by searching for shape index values S≈1, diverticula can be detected by searching for shape index values S≈−1 within the colonic wall. The curvedness values of diverticula are similar to those of polyps, although diverticula are expected to have a narrower range of curvedness values than do polyps. To extract the region of a detected single diverticulum, the detected region is expanded by morphological dilation with a spherical kernel. The size of the dilation kernel depends on the size of the detected region: large regions are dilated with a larger kernel than that used for small regions.

To reduce false-positive detections of diverticula, all air voxels were thresholds within the colon (CT value<−700 HU). Then, a morphological opening of 2.5 mm was applied to the thresholded region. Only the voxels within the detected diverticula that were not part of the air region were retained. Finally, the remaining detections that were smaller than 5 mm or larger than 10 mm were eliminated. FIG. 4B shows examples of computer-detected diverticula.

It should be noted that a detailed quantitative analysis of the performance of this detection method was not performed; instead, the detected regions were inspected visually. The visual assessment indicates that the method detected all of the visible diverticula, although it detected some false positives due to cup-like regions appearing at bases between folds which connect smoothly to the underlying colonic wall. These regions remained false positives because of the relative simplicity of the false-positive reduction method described above. Therefore, a more elaborate method would be required to achieve a clinically acceptable performance, which is left for a future study. Nevertheless, highlighting both true- and false-positive diverticula is important for the purposes of the visualization scheme presented in this study, because both types of detections are very different in shape from polyps, and thus they are useful for indicating regions that are unlikely polyps. For generating shape-scale signatures, it sufficed to select manually 10 examples of computer-detected diverticula.

Use of Computer-Simulated Lesions

The computer-simulated lesions were generated in a 384×384×512 volume. The isotropic voxel resolution of the volume was assumed to be 0.7 mm, which is the average image resolution in our clinical CTC database. The approximate range of the shapes and sizes of the simulated polyps, diverticula, and folds were determined based on actual lesions in the 14 cases containing polyps in the CTC database.

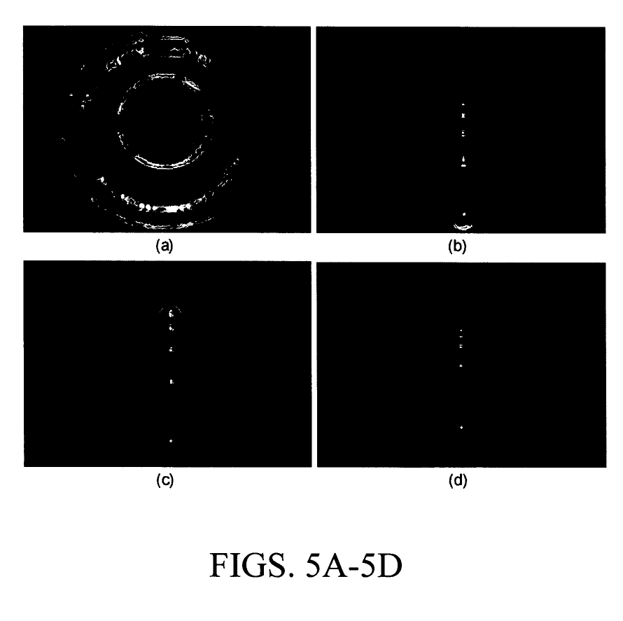

The colonic lumen was simulated by a cylinder 1.5-7.0 cm in diameter. The surface of the cylinder represented the surface of the colonic wall and was assigned the default background color. Folds were simulated by placement of several overlapping solid spheres or ellipsoids along the boundary of the simulated colonic wall in the axial plane. Seven thick spherical folds and 7 thin ellipsoid folds with diameters of 3.5-21.0 mm were simulated. Sessile polyps were simulated by placement of the center of a solid sphere or ellipsoid at the boundary of the colonic wall, and pedunculated polyps were simulated by placement of the whole sphere or ellipsoid on the boundary of the colonic wall. In each case, 7 polyps with diameters of 2.8-21.0 mm were simulated, resulting in a total of 28 simulated polyps. A diverticulum was simulated by placing of a zero-valued sphere or ellipsoid at the boundary of the colonic wall to generate a “hole” that represents the diverticulum. Seven spherical and 7 ellipsoid diverticula with diameters of 2.8-7.0 mm were simulated. To simulate the blurring of the clinical data acquired with a CT scanner, we applied a Gaussian smoothing filter with σ=2.0 to the volume. The smoothing also reduces the artificially sharp edges that could appear within the simulated objects. FIGS. 5A-5D show endoscopic views of the simulated lesions.

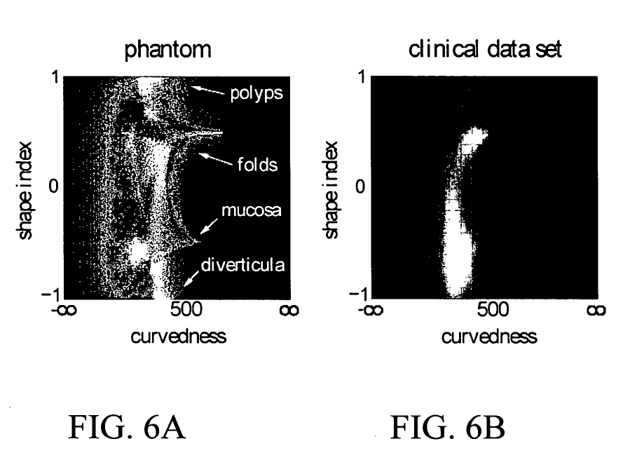

FIGS. 6A and 6B shows two shape-scale spectra. The spectra show in FIG. 6A was calculated from the computer-simulated lesions, and the spectra shown in FIG. 6B was calculated from a clinical 3-D dataset. The shape-scale spectrum of the computer-simulated lesions has two sharp peaks in the high end of the curvature values, indicating the presence of rut- or ridge-like structures. These patterns are less evident in the shape-scale spectrum of the clinical data set, indicating that the actual colonic lesions may be smoother than some structures in the simulated data. The shape-scale spectrum of the clinical data appears to be dominated by the normal colonic structures, such as folds and the colonic mucosa, over those of the few polyps and diverticula.

The Shape-Scale Color Map: Characteristic Signature and Color Interpolation

In practice, for obtaining 2-D color maps, the original shape-scale signatures may not be appropriate for the coloring of the shape-scale spectrum. This is because the shapes and scales of the different types of lesions may share common elements, and thus the shape-scale signatures of the different types of lesions may overlap.

To determine representative, but non-overlapping shape-scale regions for each type of lesion for use in the generation of color maps, the characteristic signature Sᵢ of the shape-scale signature for each lesion type i is determined. A characteristic signature is a subregion of the shape-scale signature that contains the most frequently occurring combinations of the local shape index and the curvedness values for the type of lesion that the shape-scale signature represents. Once the characteristic signature has been determined, it is assigned the unique color lᵢ that was chosen to represent lesions of type i. The points on the shape-scale spectrum that are not colored in this manner are assigned a default background color. On the other hand, the points in the transition region between a characteristic signature and the background region are colored by use of color interpolation, as described below.

Let (I¹,I²,I³) represent the components of the color assigned to the characteristic signature Sᵢ. Although these could be the red, green, and blue components of the RGB color space, the use of the RGB space in color interpolation may result in false colors. For a precise result, the color interpolation was performed in the YCᵣC_{b} (YUV) [37,17] rather than the RGB color space.

Let Dᵢ(c,s) denote the shortest Euclidean distance between the boundary of the characteristic signature Sᵢ and a point (c,s) outside Sᵢ in the shape-scale spectrum. Then, each color component thcalC^{j}(c,s) (j=1,2,3) of the color at (c,s) is calculated as

$${C}^{j} ( c , s ) = {B}^{j} + {\sum }_{n = 1}^{R} \frac{{I}_{n}^{j}}{{k}_{n} {T}^{2} {{D}_{n} ( c , s )}^{2}} \times \frac{{\sum }_{m = 1}^{R} {D}_{m} ( c , s ) - {D}_{n} ( c , s )}{{\sum }_{m = 1}^{R} {D}_{m} ( c , s )} , \qquad ( 7 )$$

where R is the number of the different types of lesions to be differentiated by the coloring scheme, B^{j} is the magnitude of the color component C^{j} in the color assigned to the background, kₙ is the sharpness parameter for the color of the lesion of type i, and T is a subsampling factor. The background is considered as an undefined lesion not in i=1, . . . ,R. The sharpness parameter controls the boundary profile of the characteristic signature Sᵢ: high values of kₙ produce sharp boundaries, and low values produce smooth boundaries.

Subsampling of the shape-scale signatures occurs when T>1 (T is set to 1 if no subsampling is desired), and can be used to limit the size of the color lookup table for faster processing. For example, if the shape index and curvedness values are represented by 1,000 entries, the resulting 2-D color lookup table would have 1,000,000 entries. For a setting of T=6, only 27,778 entries would be needed.

The color interpolation scheme provides an effective parameterization of the color map construction, and it can also be used for controlling the overlap between the shape-scale signatures. The important parameters are the sharpness parameters of the characteristic signatures and the set of the unique colors assigned to the characteristic signatures. The effects of these parameters are described below.

To implement the color map scheme, the occurrence frequencies of the local shape index and curvedness values within the shape-scale spectrum were first scaled linearly to [0,255] for each lesion type i. Then, to obtain the characteristic signature, each shape-scale signature was thresholded at the level of 84 (33% of maximum value). To limit the size of the color lookup table, the 2-D color map was subsampled by setting of the subsampling factor to T=6 in Eq. (7).

Shape-Scale Color Map: Coloring of Colon

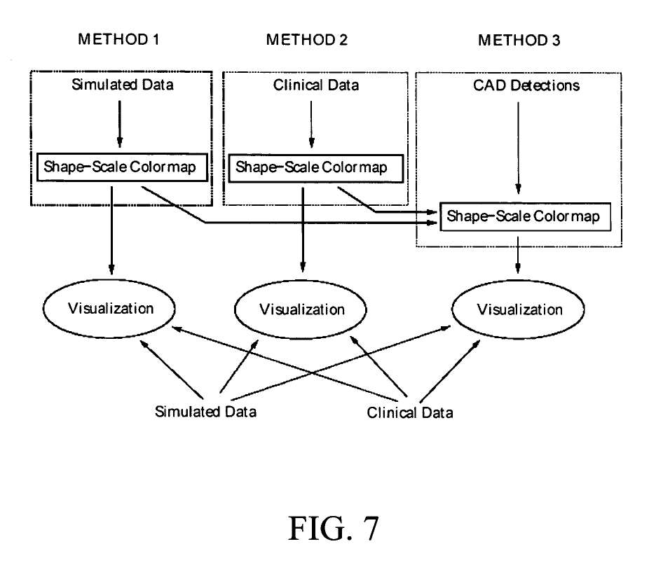

FIG. 7 shows three methods of coloring of the colon by use of the shape-scale color maps. In the first method, the characteristic signatures from the computer-simulated lesions were derived. In the second method, the signatures were derived from the lesions detected and extracted by the computer from clinical data. In both of these methods, the shape-scale color map was obtained as described above, and then it was applied to CTC cases by assigning each voxel p the color that appears at the location indicated by the shape index and curvedness values S(p) and C(p).

In the third method, called the hybrid shape-scale coloring method, the shape-scale color map information is combined with the output of our CAD scheme. Although the coloring of the colon by the first two methods enhances the lesions of interest, it is based only on the shape or scale of the lesions. Thus, false positives, such as stool that have a polyp-like shape, may be color-enhanced as well. However, our CAD scheme for polyp detection includes an effective method for the elimination of false-positive detections by an analysis of, for example, the internal texture of the polyp candidates [35]. As a result, the scheme generates only a few, if any, false-positive polyp detections for a given data set. Thus, in the third shape-scale coloring method, we first color the colon by use of either of the first two coloring methods. Next, the regions extracted by the CAD scheme, such as polyps or diverticula, are colored with pre-specified colors. The resulting coloring of the colon is expected to delineate the lesions of interest more precisely than does the application of the first two coloring methods.

FIG. 34 illustrates the steps in a method of displaying volumetric image data of a target organ as a visual aid in detecting at least one abnormality in the target organ.

In step 3401, a colormap is determined by assigning a color to each possible combination of the at least two geometric feature values. The geometric feature values are, e.g., the shape index and the curvedness index, which can be computed at each voxel in a three-dimensional data set. As described above, each assigned color corresponds to a different region type (e.g., polyps) within the target organ.

In step 3402, the volumetric image data of the target organ is obtained. The three-dimensional volumetric image data includes a plurality of voxels having a respective plurality of image data values.

In step 3403, at least two geometric feature values at each voxel in the plurality of voxels are determined based on the plurality of image data values. For example, the shape index and the curvedness index are calculated at each voxel defining the volumetric image data.

In step 3404, a display color for each voxel in the plurality of voxels is determined based on the determined colormap.

Finally, in step 3405, a color image representing the volumetric image data of the target organ is displayed based on the determined display color for each voxel in the plurality of voxels. As described above, a radiologist can use the color image to identify abnormalities in the target organ. Moreover, a CAD method may be used to identify abnormalities in the target organ, which are then displayed in a predetermined color.

FIG. 35 illustrates a system for displaying volumetric image data of a target organ as a visual aid in detecting at least one abnormality in the target organ. The volumetric image data acquisition unit 3520 is configured to obtain volumetric image data, which may be stored in organ images database 3501. The source for the volumetric image data, which may be obtained from cross-sectional image data, may be any appropriate image acquisition device such as an X ray machine, CT apparatus, and MRI apparatus. Further, the acquired data may be digitized if not already in digital form. Alternatively, the source of image data being obtained and processed may be a memory storing data produced by an image acquisition device, and the memory may be local or remote, in which case a data communication network, such as PACS (Picture Archiving Computer System), can be used to access the image data for processing according to the present invention.

The image segmentation unit 3530 is configured to extract a set of voxels from the total scanned volume, forming a database of voxels representing the segmented target organ, which is stored in the segmented organ database 3502. Abnormalities may be detected using the segmented image data of the target organ by the abnormality detection unit 3540 based on features of the image data determined by the feature calculation unit 3550. Data regarding the abnormalities may be stored in the detected abnormalities database 3503.

The feature calculation unit 3550 also calculates the shape index and the curvedness index at each voxel in the volumetric image data. The colormap determining unit 3520 assigns a color to each combination of the at least two geometric feature values (e.g., shape index and curvedness) calculated by the feature calculation unit 3550, as described above. The color assigning unit 3560 uses the colormap determined by the colormap determining unit 3520 to assign a color to each voxel in the volumetric image data. The display unit 3570 is configured to display a color image of the volumetric image data according to the colors assigned by the color assigning unit 3560.

CAD Workstation for Polyp Detection

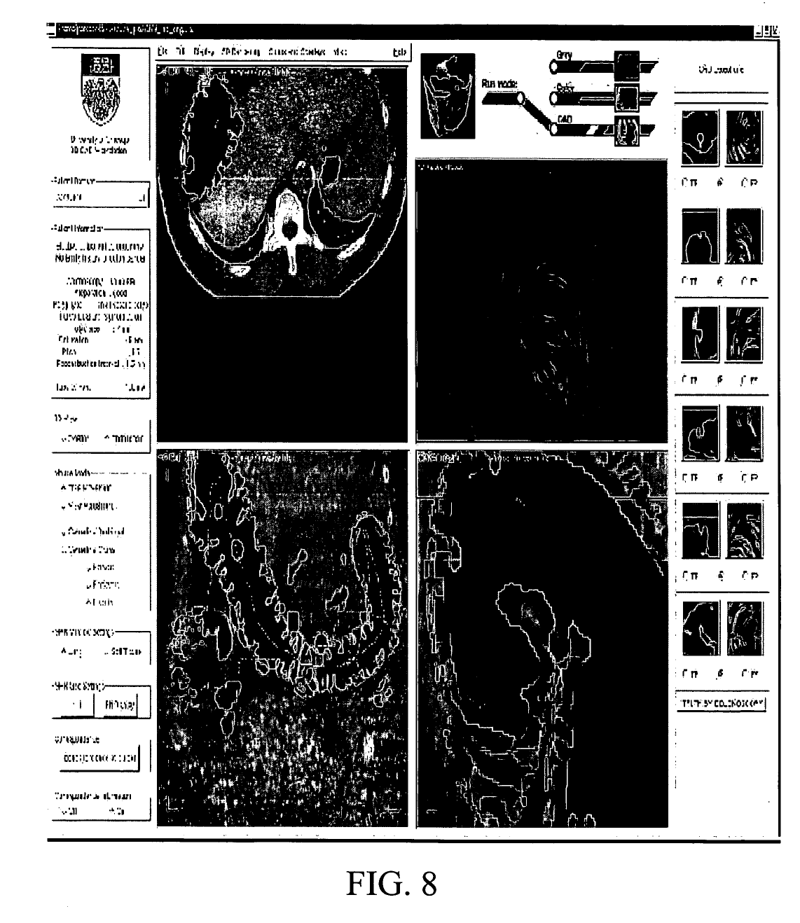

To examine the effect of the shape-scale color maps in the coloring of CTC data sets, the color map scheme was implemented in a CAD workstation for the detection of polyps in CTC (FIG. 8). The CAD workstation takes the CT images of a CTC examination as input, reconstructs a 3-D isotropic volumetric data set from the images, and performs an automated detection of the colonic polyps [33,35] on the volume.

The workstation displays the reconstructed volumetric data sets with two types of display modes: 2-D multiplanar reformatted (MPR) views including three views perpendicular to the axial, coronal, and sagittal planes of the volumetric data, and a 3-D endoscopic view that displays the colonic lumen with a perspective view. The users can examine the data set by scrolling through the images in the MPR views, by zooming into regions of interest, by manipulating the grey-scale display range, and by adjusting the 3-D view at will. The output of the detection of polyps is provided as a list of suspicious locations in a window next to the MPR views. By clicking on the list, the user can view the location of a suspected polyp on the MPR and the 3-D views.

The workstation provides these display modes with a grey-scale mode as well as with a color mode that is based on a shape-scale color map scheme. When the latter is selected, all of the visible region in the 3-D endoscopic view is colored based on the shape-scale color map, whereas in the MPR views, only the region corresponding to the colonic wall is colored with the color map.

Evaluation Method

The effect of the shape-scale coloring scheme was evaluated in two steps. In the first step, the computer-simulated lesions and 14 clinical data sets containing polyps were colored with the application of the shape-scale color maps. Several versions of the color maps were generated by adjustment of the color map parameters (i.e., the sharpness parameters and the color assignment). The effect of the color map parameters and the enhancement of the specified colonic lesions were examined visually with the CAD workstation.

In the second step of the evaluation, an observer study was performed to examine the effect of the hybrid shape-scale coloring method in the detection of colonic polyps in CTC. In this study, 20 CTC cases collected as described above were used. For each patient, either the supine or the prone data set was used. There were 10 abnormal data sets containing 11 polyps of 5-12 mm, and 10 normal data sets with no polyps. The detection performance of the computerized detection scheme was set to a by-polyp sensitivity of 92% with 1.0 false-positive detections per data set on average. The shape-scale color map was constructed by use of a high value of the sharpness parameter (kₙ=0.5). Folds were assigned a pink color, and the background was assigned a dark red color. The regions detected by the CAD scheme were colored green.

Three radiologists examined the data sets by use of the CAD workstation. Observer 1 was a radiologist experienced in the interpretation of CTC data sets, observer 2 was a radiologist not experienced with CTC, and observer 3 was a gastroenterologist. A sequential test was used, in which an observer first reads a data set without the use of the color map, and rates the confidence level regarding the presence of at least one polyp ≧5 mm on a 0-100% scale. Next, the colon is colored by the hybrid shape-scale color map, and the observer rates the confidence level again. After the observers had examined all data sets in this manner, receiver operating characteristic (ROC) analysis [38] was used to compare the observers' performance in the detection of polyps without and with the coloring scheme. For each observer, a binormal ROC curve was fitted to the confidence rating data of each observer by the use of maximum likelihood estimation. A PROPROC program was used for obtaining a maximum likelihood estimate of “proper” binormal ROC curves from the continuous ordinal-scale rating data. The difference in the observers' performance between the first and the second confidence ratings was characterized by use of A_{z}, the area under the best-fit binormal ROC curve, when the curve is plotted on a unit square. Generally, the larger the A_{z} value (0.0≦A_{z}≦1.0), the higher the performance. The statistical difference in the observer performance between the monochromatic and hybrid shape-scale colorings was evaluated by use of the two-tailed t-test to the pair of the A_{z} values.

Visual Effect of Shape-Scale Color Maps

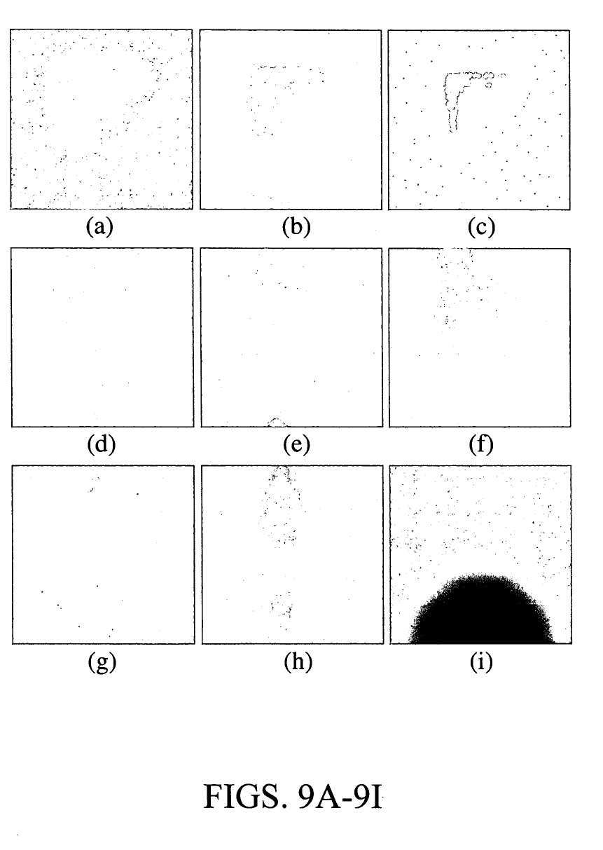

FIGS. 9A-9I show examples of the shape-scale color maps. The vertical and horizontal axes represent the shape index and curvedness, respectively. The color maps of FIGS. 9A-9F were generated by use of the computer-simulated lesions. In FIGS. 9A-9D, the polyp signature was colored blue, the fold signature was colored green, the signature of diverticula was colored red, and the background was colored grey. The color maps of FIGS. 9A-9C demonstrate the effect of the sharpness parameter kₙ in Eq. (7). In FIG. 9A, a low value of kₙ=0.02 was used, and thus the boundaries of the shape-scale signatures are blurred. In FIG. 9B, the boundaries are moderately blurred; and in FIG. 9C, a moderately high value of kₙ=0.25 results in moderately sharp boundaries. The color map of FIG. 9D demonstrates the appearance of a set of modestly saturated colors. The color maps of FIGS. 9D and 9E are similar to color map of FIG. 9B, except that different sets of colors are used. In the color map of FIG. 9E, polyps are colored red, folds are colored blue, and diverticula are colored green. The color map of FIG. 9F uses a combination of green, brown, and pink colors with a red background. The color maps of FIGS. 9G and 9H were generated by use of clinical data. The polyps are colored red and the diverticula are colored blue. In FIG. 9G, only the computer-detected polyps and diverticula were used for constructing the color map with a high value of the sharpness parameter (kₙ=1.0) for both lesion signatures. In FIG. 9H, folds were assigned a green color with a moderately smooth boundary (kₙ=0.05), and the polyps and diverticula had a sharp boundary (kₙ=1.0). The smooth boundary of the fold signature produces an overlap between the fold and polyp regions, which is colored yellow because of the color interpolation. Therefore, the colonic regions that are reminiscent of both polyps and folds will be colored yellow. This is the shape-scale region that is expected to produce most false-positive polyp detections due to folds. The color map of FIG. 9I was generated from the signature of the computer-simulated colonic wall with a smooth boundary for use with the hybrid coloring scheme. The colonic wall and diverticula are colored dark red, and the other structures are colored pink. In this case, the final coloring of the colon is obtained by coloring of the computer-detected lesions.

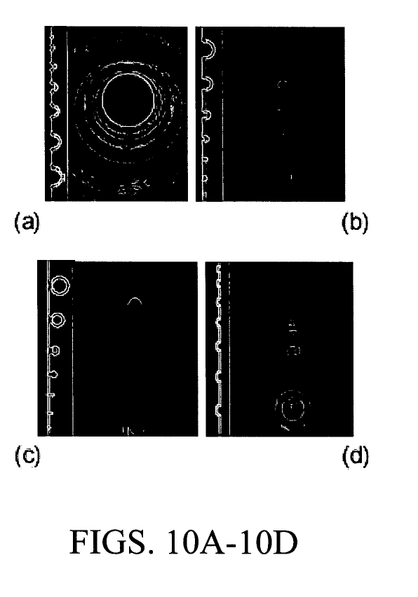

FIGS. 10A-10D show examples of the coloring of the computer-simulated lesions by use of the color map of FIG. 9B. In each figure, a cut-plane view of the surface region of the simulated colonic wall is shown to the left of the endoscopic 3-D view. As expected, the simulated folds are colored green, polyps are colored blue, and diverticula are colored red. The sessile polyps and diverticula also have a surrounding green ring at the periphery where they merge to the colonic wall, because that region is locally similar to a rut-like spinode.

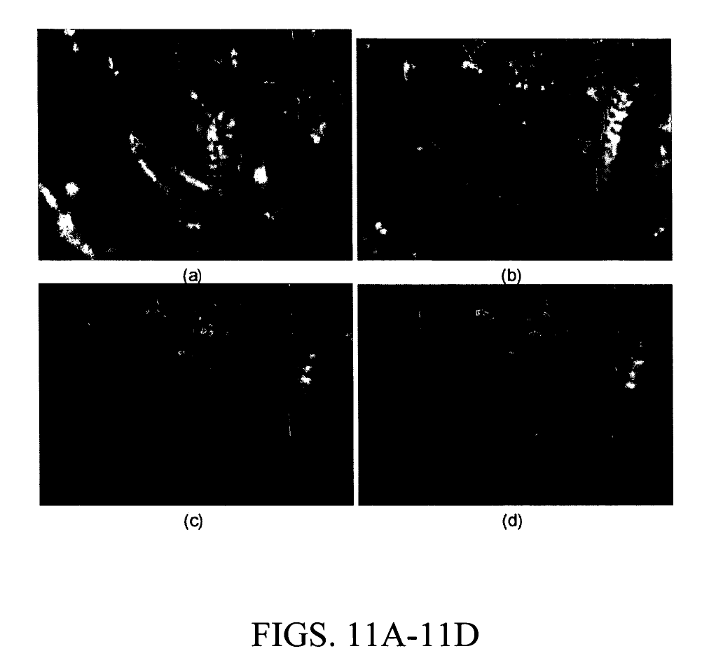

FIG. 11A shows a standard endoscopic grey-scale view of a polyp in a clinical CTC data set, and FIGS. 11B-11D show the view of FIG. 11A after the application of shape-scale color maps based on computer-simulated lesions. In FIG. 11B, the colon was colored by use of the color map of FIG. 9B. In FIG. 11C, the colon was colored by use of the color map of FIG. 9E. In FIG. 11D, the color map of FIG. 9D with modestly saturated colors was used, but the color assignment was the same as that used in FIG. 11C.

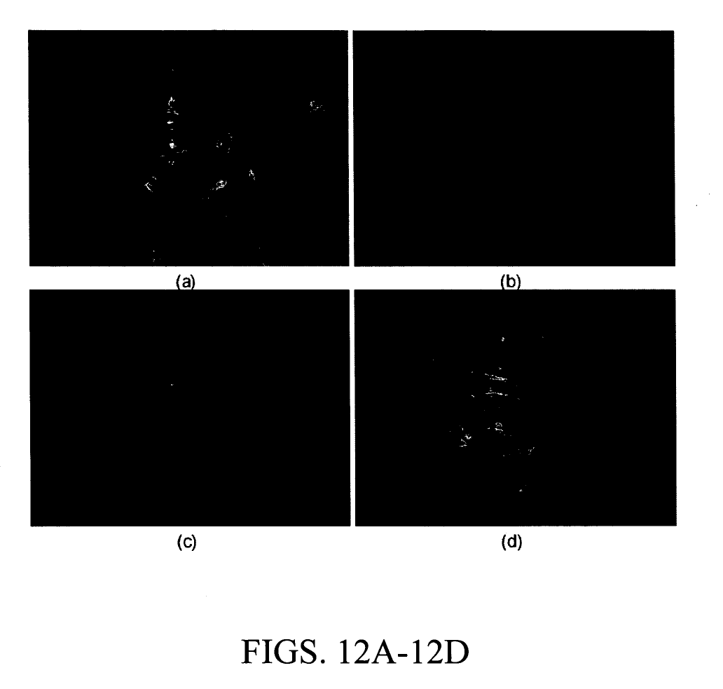

FIG. 12A shows an endoscopic grey-scale view with polyps and diverticula, and FIGS. 12B-12D show the view of FIG. 12A after the application of shape-scale color maps based on clinical data. In FIG. 12B, the color map of FIG. 9G, which was generated with a high value of the sharpness parameter (kₙ=1.0) was applied. The polyp and diverticula are colored red and blue, respectively. Other colonic structures are colored grey, but the regions similar to polyps or diverticula are associated with shades of red or blue. In FIG. 12C, the color map was constructed similarly to FIG. 12B, except that a low value of the sharpness parameter (kₙ=0.1) was used for diverticula. Thus, concave structures are colored blue more aggressively than in FIG. 12B. In FIG. 12D, the color map of FIG. 9H was applied. The mixing of red (polyp-like) and yellow (polyp- and fold-like) colors within the polyp gives it a unique appearance. The green color indicates regions that are associated only with folds.

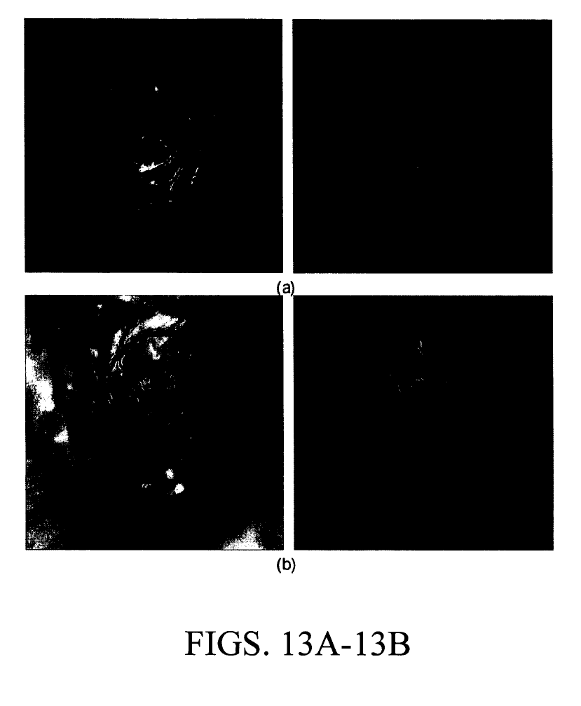

FIGS. 13A and 13B shows two examples of the application of a hybrid shape-scale color map based on the color map of FIG. 9I and the computer-detected regions. Folds are colored pink, and the background is colored dark red. In FIG. 13A, the computed-detected polyps are colored green. In FIG. 13B, the computer-detected polyps and diverticula are colored green and blue, respectively.

Observer Study

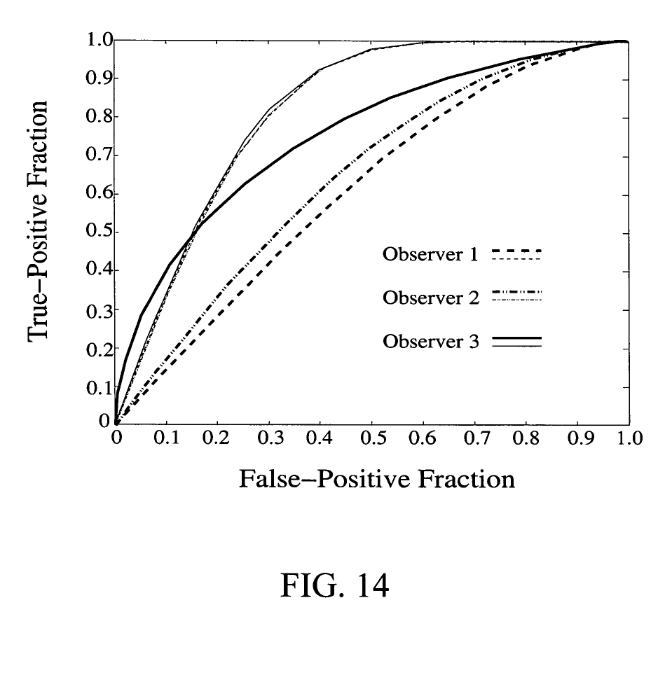

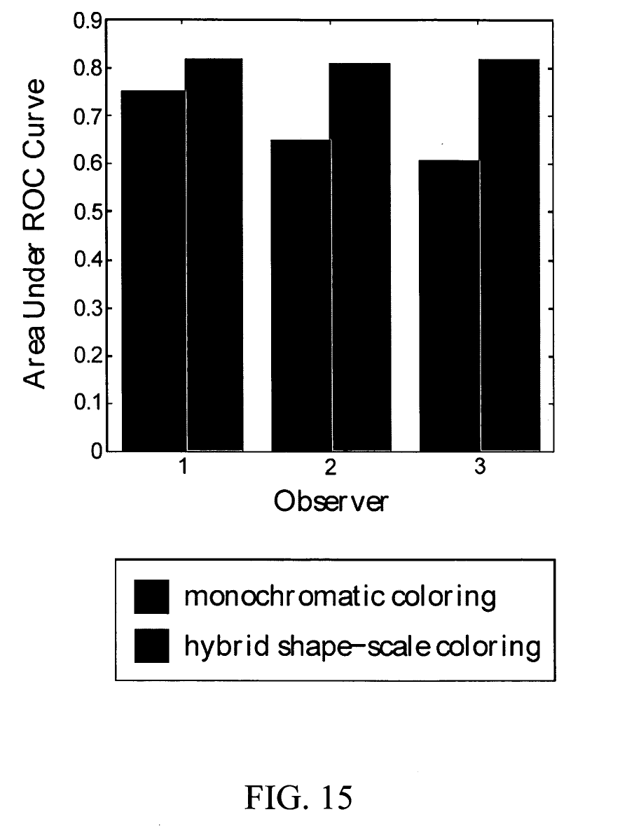

The results of the observer performance in the detection of polyps are shown by ROC curves in FIG. 14. The performance of each observer without the use of the shape-scale coloring is shown by a thick curve, and the performance of the observers with the use of the coloring is shown by a thin curve. The improvement in the observer performance yielded by the use of the shape-scale color map is demonstrated by the improved ROC curve for each observer. This improvement is also demonstrated in FIG. 15, in which each bar represents an area under the ROC curve (A_{z}) For each observer, the black bar shows the observer performance with monochromatic coloring of the colon, and the grey bar shows the performance when the colon was colored by use of the hybrid shape-scale color map.

The results indicate that the detection performance of all observers increased when the colon was colored by the hybrid color map. The two-tailed t-test for paired data showed that the difference of the A_{z} values between the monochromatic and hybrid shape-scale coloring was statistically significant (p=0.027), which confirms the usefulness of this color map. The increment was smallest for the expert radiologist (observer 1). However, the performance of the two other, inexperienced observers increased substantially and reached approximately the performance level of the expert.

The visual evaluation of the application of shape-scale color maps indicates that, as expected, colonic structures are enhanced in the colored colon. The overall perception and visibility of the lesions can be adjusted by the choice of colors. For example, the preciseness of the coloring can be adjusted by the sharpness parameter kₙ. A low value of kₙ spreads the color of lesion type i over a wider range of shapes and sizes than does a high value of kₙ that tends to color only the most typical instances of the target lesion. The shape-scale signatures from clinical data and computer-simulated data were rather similar, and the use of a low sharpness value will further increase their similarity.

The hybrid shape-scale color map construction yielded the most precise delineation of the target lesions. However, errors such as false-positive detections or incorrect segmentations of the detected lesions will show up clearly in the coloring of the colon by this approach, and false-negative detections might be colored as background if the choice of the colors is appropriate. Incorporation of the CAD data also makes this method slower than the two other methods. The observer study was also limited in the sense that only the presence of polyps in the colon was considered. The hybrid color map method appears to be the most effective approach for this purpose because the coloring of the polyps is clear and simple to interpret. On the other hand, the other color map methods may be useful for providing more versatile and rich representations of the information on colonic lumen, thereby potentially enhancing the analysis of the colon. However, an extended training period for radiologists may be required to efficiently interpret such visualizations of the colon. The analysis of the clinical benefits of the more complicated color map representations is a topic for a future study.

The results of the observer performance study must be considered preliminary because of the small number of observers (3) and data sets (20). Nevertheless, the results are similar to those obtained previously in the evaluation of CAD schemes for breast and chest imaging: the performance of a radiologist is increased when a computer aid is used, and the performance of non-experienced readers may be improved to the level of expert readers [39,40]. Therefore, one can expect similar performance results with this method when a larger database and a larger number of observers are employed. The use of the scheme has also other potential benefits that were not addressed directly by the study. If the color map visualization is available as soon as a CTC case has been loaded, the radiologist may be able to find the polyps faster than with the use of the standard screening schemes. Also, the color map visualization may boost radiologist's confidence in the diagnosis, thereby allowing the radiologist to screen CTC cases faster than with standard methods in which the visual representation of the colon can be more ambiguous.

Compared with previous work in which fully automated methods for the detection of polyps in CTC were developed, the present invention is unique because it focuses on the visualization of the colon for radiologists. Various visualization schemes are presented that can be used to color lesions of interest in the colonic lumen based on a systematic method. The required color maps may be prepared by use of either simulated or actual lesions, and the output of the CAD scheme can be incorporated into the visualization scheme by use of the hybrid coloring method. Unlike in previous studies, an observer study was provided to evaluate the effect of the coloring schemes in polyp detection by radiologists.

Flat lesions were not included in the study because the clinical database did not contain such lesions. However, although flat lesions hardly protrude from the colonic wall, they are still expected to have the local cap-like shape of a polyp, and the curvedness values of flat lesions are expected to be lower than those of typical polyps. Because the region that corresponds to flat lesions in the shape-scale spectrum is not expected to have a significant overlap with the other colonic non-polypoid lesions, the visualization of flat lesions may be implemented simply by an adjustment of the curvedness range of the polyp signatures.

A new visualization method for a rapid examination of the colon by use of virtual endoscopy was developed. The colonic structures of interest are detected and colored based on their characteristic signature patterns in the shape-scale spectrum. The method was evaluated by use of computer-simulated lesions, clinical CTC data sets, and an observer study. The results of the visual examination indicate that shape-scale color maps can be used for enhancing the visibility and conspicuity of the target lesions. The results of the observer study indicate that the performance of readers not experienced in the detection of polyps in CTC can be improved substantially by use of the hybrid shape-scale color map scheme. Combined with our CAD workstation for the detection of colonic polyps, the visualization scheme is expected to reduce radiologists' interpretation time and to improve the diagnostic performance of the detection of colonic neoplasms in CT colonography.

The present method of shape-scale signatures can also be implemented more generally by one of ordinary skill in the art for the detection of other abnormalities of other organs. In particular, the present method is applicable to any type of convex (peak-like) or concave (pit-like) abnormalities in N dimensions (N>1). Thus, an embodiment of the present method can be readily applied to 2D/3D aneurysms, embolisms, solid lung cancer (especially those that stick on the chest wall), solid stomach cancer, etc.

II. Computation of Colon Centerline in CT Colonography

According to the present invention, there is provided a method for the computation of a colon centerline that is faster than any of the centerline algorithms previously presented and that relies less than other algorithms on complete colon-segments identification. The method first extracts local maxima in a distance map of a segmented colonic lumen. The maxima are considered to be nodes in a set of graphs, and are iteratively linked together, based on a set of connection criteria, giving a minimum distance spanning tree. The connection criteria are computed from the distance from object boundary, the Euclidean distance between nodes and the voxel values on the pathway between pairs of nodes. After the last iteration, redundant branches are removed and end segments are recovered for each remaining graph. A subset of the initial maxima is used for distinguishing between the colon and non-colonic centerline segments among the set of graphs, giving the final centerline representation.

Detection of Local Maxima in DM

Given a DM of the segmented colonic lumen (air-filled region), a subset that consists of the local 6- and 4-neighborhood maxima within the DM is extracted. The size of a maximum is defined to be its DM voxel value, and the position is defined to be its voxel coordinates. Here, a 6-neighbor maximum is defined as a voxel v_{x,y,z} within the DM at which all six neighborhood voxels (v_{x±1,y,z}, v_{x,y±1,z}, v_{x,y,z±1}) have a value smaller than that of the voxel. It can be interpreted as the center of the locally largest sphere fully enclosed by the object. A 4-neighbor maximum is a voxel v_{x,y,z}.in the DM at which all neighboring voxels in two of the three 6-neighbor directions have a value smaller than that of the voxel (i.e., v_{x±1,y,z}, v_{x,y±1,z}, or v_{x±1,y,z}, v_{x,y,z±1} or v_{x,y±1,z}, v_{x,y,z±1}). A 4-neighbor maximum can be interpreted as the center of the locally largest cylindrical structure that is fully enclosed by the object.

A point at the center of a segment of the colon usually has a fully enclosed, locally largest, sphere due to the folds within the colon, or a locally largest fully enclosed cylindrical structure due to the cylindrical shape of the colon. Therefore, a good representation of a centerline can be found by extraction of the 6- and 4-neighborhood maxima. Our algorithm uses a subset of these maxima as input. The subset, M, is chosen so that, for every maximum Mᵢ in M, there is no other maximum in M that has a distance to Mᵢ less than the size of Mᵢ scaled by a constant (i.e., the seedscalefactor or regularscalefactor, see Section III).

Seedpoint Extraction

Among the set M of maxima, we select an initial set, M_{S}, of maxima in M consisting of 6-neighbor maxima with sizes larger than a user-defined minimum-size threshold value (i.e., minseedsize, see Section III). M_{S} will serve as a set of seed points for the location of the centerline. The seed points are used as a guide for determining which of the final centerline segments actually belongs to the colon. The lower the minimum size threshold value, the more sensitive to non-colonic objects the algorithm is. The higher the minimum size threshold value, the less likely it is that the full colon centerline is found. A parameter that defines the minimum number of seed points selected (i.e., minseedamount, see Section III) ensures that the algorithm will work even in cases where the colon is extraordinarily narrow, even if this implies that the minimum-size threshold value is violated.

Let G represent a set of graphs. Each maximum in M_{S} is considered to be a node in G, assigned the position of the maximum (i.e., the voxel coordinates in 3-D space) and the size of the maximum (i.e., the DM voxel value of the maximum), and removed from M. Initially, each node in G is equivalent to a graph (see FIG. 29A).

Graph Connection. Let G_{A} and G_{B} be graphs in G. A link between a node A in G_{A} and a node B in G_{B} is created, joining the two graphs, if the following three conditions are satisfied:
1) G_{A} is not the same graph as G_{B}
2) In the DM, on a straight line between A and B, no voxel with a value less than a pre-defined threshold value exists.
3) There is no other node C in G, closer than B to A in terms of Euclidean distance, which can be connected to A, fulfilling criteria 1-3.

The above criteria 1-3 are applied to all possible nodes in G. The resulting G consists of one or more graphs, each graph consisting of one or more nodes, and representing a minimum distance spanning tree within the colonic lumen (see FIG. 29B).

Window-Based Iteration

Starting with the remaining maxima in M with the largest size and iterating down to the maxima with the smallest size, maxima are removed from M and instead are considered as new single-node graphs within G (see FIG. 29C). In addition, during each iteration, the Graph Connection step is applied to all new nodes. Some of the graphs in G may grow further during this process (see FIG. 29D).

Branch Cutting

Each graph in G contains a major centerline and several additional branches, but no loops. Iteratively, for graphs G_{N} within G containing a node with more than two links, all available single-ended nodes in G_{N} (i.e., the end nodes of each branch in G_{N}) are removed. The iteration stops when no graph in G contains a node with more than two links (see FIGS. 29E and 29F).

Branch Recovery

The branch-cutting process removes not only unwanted branches, but also the end segments of the actual centerline. These unintentionally removed segments are recovered for each end node in each graph in G. For an existing end node in G, end-node candidates are defined as the nodes in all of the branches that were once connected to the existing end node, but were later removed during Branch Cutting. One of the end-node candidates is selected as the new end node. The branch, or part of the branch, that connects the existing and the new end node is recovered. The selection of the new end node is performed in the following manner: First, we measure the distance from each of the end-node candidates to the existing end node. Second, we measure the distance from each of the end-node candidates to the node with a link to the existing end node. Third, comparing the two distances measured for each end node candidate, nodes that have the longer distance to the existing end node are removed from the list of endnode candidates. This prevents a branch that turns back to the extracted centerline from erroneously being recovered as an end segment. Finally, among the remaining end-node candidates, the new end node is chosen as the end-node candidate with the largest distance to the current end node (see FIG. 29G).

Save Result

Each graph containing at least one seed point is stored as a resulting centerline segment. All other graphs are removed because a graph without a seed point is considered to be a centerline segment for an extra-colonic structure.

For evaluating the effect of different parameters, we validated our algorithm on a computer-generated colon phantom. The input is an arbitrary curve in 3-D space and the radius, R, of the colon phantom to be generated. The phantom is generated as follows: Traversing the curve given as input, at a uniform path distance of 1 voxel, compute the radius of a cylinder with thickness of 1 voxel, and position it with the main axis in the path direction and centered on the curve. The radius of the cylinder is computed as R with additional random noise with range of 2 voxels. The exception is that, at a uniform distance of 40 voxels of path length, a fold is created. A fold is created by linearly decreasing the radius to 20% of R, after which the radius is increased linearly to its initial value. The path length of the fold-decreasing and -increasing phases is 10 voxels each. At this stage, the phantom contains has the overall shape and randomness, but folds are unnaturally deep and saw-tooth shaped, and the surface is jaggy and synthetic looking. By dilation with a spherical dilation kernel with a radius of 6 voxels, the final 3-D phantom of the colonic lumen was created, with the intended look and fold sizes.

In the following, the terms “reference” and “slave” centerlines are used. The reference centerline is considered the “true” centerline. For quantifying the degree of deviation of a slave centerline from a reference centerline, primarily two types of quantities were calculated.

Displacement for a single point on the reference centerline is defined as the shortest Euclidean distance to the slave centerline with a straight path within the lumen, if existing. Otherwise, displacement is not defined for the point. For a reference centerline, mean displacement is defined as the average of all displacements along the centerline, max displacement is defined as the largest of all displacements along the centerline, and standard deviation is defined as the standard deviation of all displacements along the centerline. The centerline extension is the fraction (computed as percentages) of the number of defined displacement points when compared to the number of both defined and undefined displacement points.

For a set of centerlines, the average extension, the average of the maximum, the average of the mean, and the average standard deviations of the slave centerline when compared to the corresponding reference centerlines, are the averages over all cases of the centerline extensions, max displacements, mean displacements, and standard deviation, respectively (see FIG. 30).

The centerlines generated by the algorithm presented, were always regarded as being the slave centerlines in the phantom studies.

For evaluating the accuracy of the centerline generated by our algorithm on clinical cases, a total of 20 CTC cases were selected from CTC examinations performed at our institution between 1997 and 2000. All patients underwent CTC after a precolonoscopy preparation of barium enema bowel cleansing, by use of 48-hour liquid diets and agents such as polyethylene glycol, magnesium citrate, or bisacodyl and phospho soda. Then a soft-tip tube without a training cuff was inserted into the rectum, and the colon was gradually insufflated with room air until the patient experienced mild discomfort (approximately 40 puffs or 1 liter). CT scanning was performed by use of a helical CT scanner (GE9800 CTi and LightSpeed QX/i; GE Medical Systems, Milwaukee, Wis.) with 2.5 mm to 5 mm collimation, and reconstruction intervals of 1.5 mm to 2.5 mm. The matrix size of the resulting axial images was 512×512, with a spatial resolution of 0.5 mm/pixel to 0.7 mm/pixel. A reduced current of 60 mA or 100 mA with 120 kVp was used for minimizing the radiation exposure. CT scanning was performed in both supine and prone positions, and thus 20 cases included a total of 40 CTC scans (i.e., 20 supine and 20 prone CTC scans).

Although the colon was cleansed and distended for all of the patients, the optimality of the cleansing and distension varied among patients, and thus some of the colons had residual stool and fluid as well as non-distended segments of the colon, as often reported by other CTC studies [20]. To evaluate the robustness of the centerline algorithm under a wide spectrum of conditions, we included cases with feces, fluid, a poorly distended colon, and motion artifacts in the 20 cases as described below: (1) 20 CTC scans, classified as “straightforward,” had a well distended colon, with only a small number of narrow sections and thin colonic walls; (2) 10 CTC scans, classified as “moderate,” contained narrow and collapsed regions and thin walls; and (3) 10 CTC scans, classified as “challenging,” had a poorly distended colon with thin walls, and a large number of collapsed regions due to residual stool, fluid, and spasm. Also, motion artifacts were present in some of the cases.

Generally, the segmented cases had a large number of fragments of small bowel, lungs, and stomach remaining in the segmented colon. No case was completely free of extra-colonic components (see FIG. 31A-31I).

Three radiologists, A, B, and C, manually drew, independent of each other, a centerline for the colon in each of the 40 scans by using in-house 3-D computer software with a custom centerline tool. They were instructed to include all non-collapsed parts of the colon.

Implementation Issues

The following implementation details and settings are used in our implementation of the centerline algorithm. They serve as a guide for efficient implementation, but are not proposed to be the only way for implementing the algorithm.

DM. We use the chamfer 3-4-5 DT [21] for computing the distance map.

Seedscalefactor, Regularscalefactor. Scale factors that scale a spherical neighborhood of a seed point and regular point, respectively, used for removing other maxima within a local neighborhood. If the values for these parameters are set too high, maxima that happen to be close in 3-D space, but are located in different parts of the colon may accidentally be removed. If the parameters are set too low, the computation time increases without any increase in the accuracy of the resulting centerline. In our implementation, the size of the sphere for a seed point (seedscalefactor) is set to 1.5 times the physical sphere radius, as indicated by the DM value at that point. The value was chosen in order for the spherical neighborhood it represents to cover the colonic lumen including parts of the wall. The size of the sphere for a regular point (regularscalefactor) is set to 1.1 times the physical sphere radius. The seed points are considered more accurately positioned in 3-D space than the regular points, hence the larger value for seedscalefactor. Additionally, we set a minimum radius to 10 voxels for any sphere. This is not necessary for the algorithm to work. However, it will give a slight speed improvement.

Minseedsize. Smallest maxima size that will be regarded as seed point. It is typically set to a size within the range of 50-80 for 3-4-5 DT, but this may vary depending on the resolution of the colon in CTC scans. minseedsize should reflect the radius in voxels for the well-distended parts of the colon.

Minseedamount. Minimum number of seed points desired. This parameter may, if necessary, override minseedsize. It is typically set within the range of 30-70. It may be set to zero in order to reduce the number of parameters. The parameter ensures that no less than minseedamount seed points are selected, even if this implies that the parameter minseedsize must be overruled. The parameter is useful only for unusual cases in which a large portion of the colon is collapsed. In such a case, if this parameter is not used (i.e., set to zero), minseedsize would have to be lowered for all cases, which increases the risk of finding the centerline of non colonic structures.

Nondt. Highest voxel value in DM considered to be outside the colonic lumen. We typically use a nondt value of 3 for 3-4-5 DT. If Euclidean DT is to be used, a value of 1 would be appropriate. The partial volume effect can create holes between neighboring colonic walls, and thus a higher value of nondt can avoid the generation of centerlines through the holes. However, setting too high a value for nondt may prevent the extraction of the entire colon centerline. The parameter should be set considering the scale of the DT used to reflect correctly how many layers it peels off the surface in the DM.

Detection of Local Maxima in DM. The removal of close-by maxima can be implemented effectively in a two-step manner: First, store all maxima found in DM in a look-up table (LUT). Also, set up an empty (all zero) volume Vₘ with the same dimensions as those of the DM. Second, starting with the largest maxima in the LUT, for each maximum Mᵢ check whether the corresponding voxel in Vₘ is zero. If so, set all voxels in Vₘ within a desired radius (i.e., the DM value of Mᵢ scaled by seedscalefactor or regularscalefactor) to value 1. If not, remove Mᵢ from the LUT.

Graph Connection. It is important to evaluate the iteration on only the recently added nodes in G (i.e., the nodes that have not previously been evaluated by Graph Connection). In addition, the iteration must not re-visit a node before visiting the next node in the list. Otherwise, the computation time for the Graph Connection process could easily take several minutes. It should be noted that we implemented the path search with a simple Bresenham line algorithm that checks the voxel values for all voxels on the path. If any non dt voxel is found, it returns FALSE, but returns TRUE otherwise.

Results: Evaluation Based on Colon Phantom

Two phantom studies were made. In the first study, a centerline was computed for phantoms with different radius, ranging from 3 to 30 voxels (2-21 mm). As seen in Table I, the centerline computation time is nearly constant (2.3-2.9 seconds), and the extension drops from 100% only in very narrow (3-5-voxel radius) and very well-distended cases (30-voxel radius). For narrow passages, this is because the centerline is split into multiple segments due to sharp turns in the colonic pathway. For a large radius, the decrease occurs because the ends of the original and computer-generated segments are not perfectly aligned. Thus, the similarity computation algorithm will regard these segments as missing. With increased phantom radius, the average displacement-related parameters (i.e., the mean displacement, the max displacement, and the standard deviation) of the computed centerline increases when compared to the path used for creating the phantom. This is because, when the phantom radius becomes large, at sharp turns close-by colonic walls will merge, thus shifting the actual central path of the colon from its original position (see FIGS. 32A-32D). As expected, with increased radius the number of maxima (both 6N and 4N) detected in the DM increases significantly. The algorithm successfully decreases the amount to a close to constant number of maxima (59-95) used.

In the second study, the seedscalefactor was varied within the range of 0.1-2.0. The regularscalefactor was set to 0.7×seedscalefactor. The phantom radius was set to 15 voxels. As seen in Table II, all of the colon centerline was computed regardless of the seedscalefactor. Also, the average displacement-related parameters (i.e., the mean displacement, the max displacement, and the standard deviation) are close to constant. The computation time was close to constant except for the smallest scale factor, when the time consumption increased dramatically. The reason for this is that with such small scales, almost every possible maximum found in the DM will be used by the algorithm (8200 out of 8275). It should be noted that the creation of the graph structure is not linear but, for n nodes, in the worst case proportional to (n/2)². This is because every node added must be compared to all existing nodes in the graph structure. The complexity of the remaining parts of the algorithm is proportional to the number of voxels in the volume data set or to the number of nodes in the graph structure.

To estimate whether a complexity of (n/2)² will interfere with the performance of the algorithm under normal circumstances, we compute a reasonable upper estimate of the number of maxima picked up by the algorithm by using the following model: Assume that a colon C is cylinder-shaped, approximately 1500 mm long, and has an average radius of 25 mm [22]. Assume also that we have an isotropic voxel space and 1 voxel=1 mm³. C will consist of V_{c}=π25²×1500≈2.94×10⁶ voxels. Each maximum used by the algorithm will be the single maximum within a spherical neighborhood, and occupy vₘ=(⅓)×4πr³ voxels where r is the shortest distance to the object boundary. This implies that the seedscalefactor and the regularscalefactor parameters are both set to 1.0. If the number of maxima collected is the inverse of the squared maxima radius, and maxima radii are, on average, 15 mm, an upper limit of the total number of maxima used will be

$$m = \frac{{v}_{c}}{{\sum }_{r = 1}^{30} \frac{4 π {r}^{3}}{3 {r}^{2}}} \approx \frac{2.94 \times {10}^{6}}{\frac{4 π}{3} 30 \times 15} \approx 1500.$$

As seen in Table II, column 2, the centerline can be computed in less than 3 seconds for this number of maxima, which indicates that the computation time is not likely to become large for any colon.

Results: Evaluation Based on Clinical Cases

The computer-generated centerlines for the 40 CTC scans were always regarded as being the slave centerlines when compared to the human-generated centerlines. FIGS. 33A and 33B show an example of a 2-D projection of a computer-generated centerline and its displacement at each location in 3-D when compared to a reference centerline generated by a radiologist. In addition, for each CTC scan, the centerlines generated by the radiologists were compared with each other. This comparison was performed for all permutations of the centerlines drawn by A, B, and C with respect to both reference and slave centerlines.

Table III shows the results of the comparison between computer-and human-generated centerlines, and between human-generated centerlines. As shown, all quantities for the computer-generated centerline, except for the extension, are comparable with or even better than the pair-wise comparison between human centerlines. The average maximum displacement was relatively large for all cases, both when human centerlines were compared and when human centerlines were compared with the computed centerline. However, most of the large displacements were observed in the end regions of either the cecum or rectum. In these regions, it was difficult to determine the centerlines uniquely because of the large and irregular shapes of the regions.

Table III also shows that the extension of the computer generated centerlines, on average, was 92% of the human-generated centerlines, comparable to 94% when human-drawn centerlines were compared with each other. By computation of the same quantity for the three groups (straightforward, moderate, and challenging) separately, it is shown in Table IV that, for the straightforward cases, the average extension for both computer-generated centerlines and human-drawn centerlines were close to 100% of the entire colon (97% and 98%, respectively). For the moderate cases, the average extension of the computer-generated centerlines was lower than that obtained from the straightforward cases (91% and 97%, respectively). For the challenging cases, the extension of the computed centerlines was, on average, 81% of the colon, compared to 84% extension by the human-drawn centerlines. The computer generated centerlines covered less of the colon relative to the human-generated centerlines because some small, very narrow segments of the colon were found by the radiologists, but were missed by the algorithm. This was expected because these narrow segments were considered to be non-colonic objects by the algorithm. However, even the centerlines drawn by radiologists had poor extension when compared to each other. This is mainly for the same reason as above: the challenging cases often contained a large amount of stool, collapsed regions, and extra-colonic findings which made it difficult for the radiologists to find the true colonic path. Thus, the centerlines drawn by each radiologist covered different parts of the colon.

A two-sample t-test was performed to evaluate the difference in the displacements between human-drawn centerlines and between human-drawn and computer-generated centerlines. The null hypothesis was that there was no difference. To explore this hypothesis, all displacements between human-drawn centerlines used for computing the averages in Table III, were pooled and compared to those of all displacements between human-and computer-generated centerlines. The null hypothesis was not rejected at the =0.05 level. This result indicates that there was no statistically significant difference in the displacements between the human-drawn centerlines and between human-drawn and computer-generated centerlines. However, the average extension of the computer-generated centerline was statistically significantly less than that of the human-drawn centerlines.

For evaluating the speed, we measured the CPU time for computing the centerlines. We did not include the calculation time for the interpolation of the input data or for the segmentation of the colon. Using an Intel Pentium-based 800 MHz computer with 512 MB memory, for the 40 scans, the centerline computation time was in the range of 2.6-7.4 seconds, with an average of 4.8 seconds. The distance transform used in this study was computed, on average, in 5.7 seconds, giving a total centerline computation time of 10.5 seconds, on average, for the 40 CTC scans.

In 4 of the 40 cases, the computer-generated centerline contained a total of 5 centerline segments that did not correspond to any segment of the centerline drawn by a human observer. These segments respectively represented 0.9%, 1.0%, 1.4%, 1.6%, and 6.7% of the total centerline, within a path length range of 18-148 mm.

Visual inspection confirmed that, in no case, the computer generated centerline went through any hole created by the partial volume effect or its equivalent.

The proposed centerline algorithm has several advantages. First, to our knowledge, it is the fastest implementation among the methods for computation of colon centerlines reported in the literature.

Second, our algorithm performs a global search for selection of seed points, without any human intervention. The global search is designed to find seed points in all of the distended segments of the colonic lumen even when these segments are disconnected. Therefore, the algorithm can generate centerlines without being trapped by collapsed regions.

Third, the algorithm is robust to segmentation of the colon of non-optimal quality, with collapsed regions, and stool within the colonic lumen; it computes all available centerlines, regardless of the location and length of individual segments. The set of globally extracted seed points determines which segments belong to the colon.

In addition to these advantages, the algorithm has an important feature. It uses 6- and 4-neighbor maxima of the DM as a guide for positioning the centerline. These maxima are, by definition, well centered in the DM, and thus the resulting centerline is also centered.

The main reason for the speed of the algorithm is data reduction. Initially, a set of maxima representing the centerline pathway is extracted from the DM, reducing the amount of data by a factor of 100,000 compared to the DM. Only the reduced data set is used for creation of the final centerline. The fact that the algorithm does not depend on a specific type of DT, but can be used with an integer-based DT, reduces the total computation time even further [21].

Because the algorithm attempts to find segments of the centerline in different parts of the abdomen independently, application of the algorithm to poorly segmented colons may generate centerline segments for non-colonic structures, such as the small bowel and the lungs. In most of these cases we observed that the entire colon centerline was extracted and could easily be identified as being the longest segment. However, if only parts of the entire colon centerline were generated, anatomy-based post-processing might be necessary for the centerline segments to be connected correctly. This, however, is beyond the scope of the present study.

Several criteria were evaluated for recovering the end segments of a centerline in the Branch Recovery step. Generally, long branches appear to be a good representation of the lost end segments of the centerline. It is, however, not guaranteed that this gives the “correct” end segments. Our method described in Section Branch Recovery was able to recover the most extended branch that does not turn back to the extracted centerline, and thus found to be most appropriate method.

As shown in Table III and IV, for all centerline comparisons, the average maximum displacement was at least 15 mm. Such a large deviation often occurred at the end points. This can be explained in terms of uncertainty. That is, neither the radiologists nor the computer algorithm knew exactly in which direction the end point should be, not even whether the centerline was supposed to be drawn all the way to the colonic wall indicating the end of cecum, rectum, or an in-between segment, or to stop somewhat earlier.

As stated above, the computed centerlines, on average, yielded slightly less extension compared to the human generated centerline because narrow segments between collapsed regions were sometimes completely missed by the algorithm. This was expected, because the algorithm could not find seed points anywhere within such a narrow region, and thus the segments were not regarded as part of the colon. It is possible to lower the minimum-size threshold value for selection of seed points to find these narrow segments although it may potentially increase the number of extra-colonic centerlines.

It should be noted that it do exist centerline algorithms which can produce centerlines that are, on average, more well centered with respect to a DM than the algorithm presented in this paper. The reason for this is, the centerline is based on a set of discrete, centered, nodes within the colon. In cases where the Euclidean distance between connected nodes is large, the in-between connection might be off the central path.

III. Detection of Colorectal Masses in CT Colonography

Because the mass detection methods were designed to be integrated in the inventors' previously developed automated CAD scheme for the detection of polyps, a brief overview of that scheme is provided. The specific details are presented elsewhere [33,34,35,36,49].

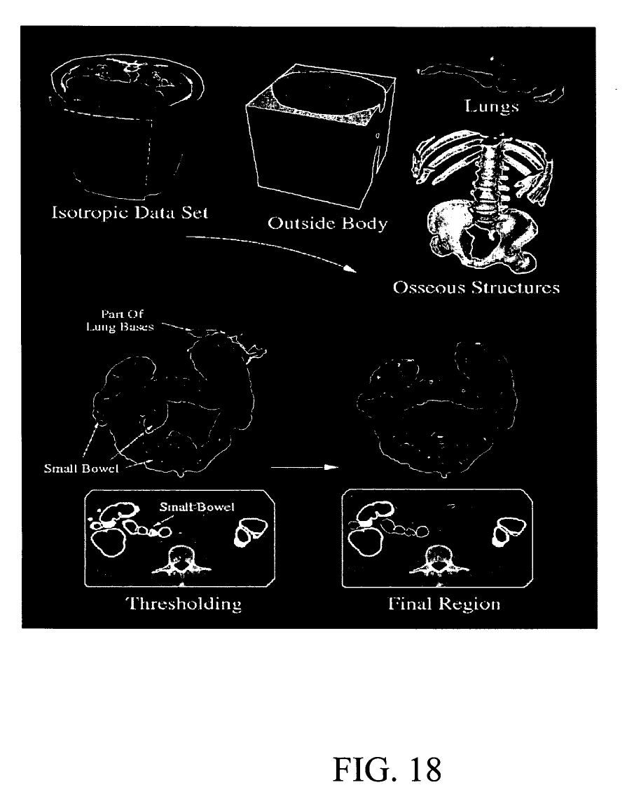

Given a 3-D isotropic CTC data set, a thick region encompassing the colonic wall is extracted automatically by use of a knowledge-guided segmentation technique [23]. In this technique, the region outside the body, the osseous structures, and the lungs are first eliminated from further analysis by use of histogram analysis, thresholding, and morphological operations (FIG. 18). The region of the colonic wall is extracted from the remaining region by thresholding of the CT and gradient values. Because the extracted region may contain extra-colonic components, such as small bowel, a self-adjusting volume-growing step is used to identify the colonic lumen based on the initially extracted region. The final region of the colonic wall is obtained by addition of surface layers to the volume-grown region of the colonic lumen. On average, 98% of the visible colonic wall is covered by the extracted region, and approximately 10-15% of the extracted region is associated with extra-colonic structures [36].

The regions with polyp-like characteristics are detected from the extracted region of the colonic wall by use of hysteresis thresholding [33,57]. The hysteresis thresholding is guided by two volumetric features called the shape index (SI) and the curvedness (CV) [29], which can be defined as

$$\begin{matrix}SI ( p ) = \frac{1}{2} - \frac{1}{π} arctan \frac{{k}_{1} ( p ) + {k}_{2} ( p )}{{k}_{1} ( p ) - {k}_{2} ( p )} , & ( 8 ) \\ CV ( p ) = \frac{2}{π} ln \sqrt{( {{k}_{1} ( p )}^{2} + {{k}_{2} ( p )}^{2} ) / 2 ,} & ( 9 )\end{matrix}$$

where k₁(p) and k₂ (p) are the principal curvatures of the iso-surface passing through voxel p [29]. The SI classifies the topological volumetric shape at a voxel, and the CV characterizes the scale of that shape. To implement the hysteresis thresholding, one first locates the voxels that have SI and CV values within SI∈[0.9,1.0] and CV∈[6.68,8.52]. The regions represented by such voxels are expanded by addition of 26-connected boundary voxels that have their SI and CV values within SI∈[0.8,1.0] and CV∈[6.40,8.52]. The details of this technique have been presented elsewhere [33].

The regions extracted by hysteresis thresholding, or initial detections, do not necessarily cover completely the visually perceived region of the detected lesions. For analyzing the internal structure of the detections for FP reduction, the final regions of the detections, or the polyp candidates, are extracted by use of a conditional morphological dilation technique [35]. In this technique, a detected region is expanded within the colonic wall by morphological dilation, and the dilation step for which the growth rate of the expanding region is smallest is chosen to represent the final region of the detection [35].

Finally, FP polyp candidates are reduced by use of a quadratic classifier based on quadratic discriminant analysis. The classifier is trained by labeling of each polyp candidate as a true-positive (TP) or FP detection based on the feature values calculated for the candidate region. The quadratic classifier then generates a decision boundary that partitions the feature space by use of a hyperquadratic surface. The result of the partitioning can be represented by a discrimination function, which projects the multi-dimensional feature space into a scalar decision variable. For a polyp candidate, the value of the decision variable determines whether the candidate belongs to the TP or FP class [33]. The polyp candidates in the TP class yield the final set of the polyps detected by the CAD scheme.

Overview of Mass Detection

The first method for mass detection, fuzzy merging, identifies intraluminal masses that protrude from the colonic wall into the colonic lumen and is described below. The second method, wall-thickening analysis, detects non-intraluminal types of masses that appear as an abnormal thickening of the colonic wall. This method is described in more detail below. Once the locations of the mass candidates have been established, we a level set method is used to extract the complete regions of the mass candidates, as described below.

To reduce FP candidates, shape and texture features were calculated from the detected mass regions. A quadratic classifier discriminates the mass candidates into TPs and FPs as in the case of polyp candidates. However, the classifier is trained separately for polyps and the two types of masses, because these lesions have considerably different feature characteristics. Once the final candidates are established, the polyp candidates that overlap the regions of mass candidates are eliminated, because such polyp candidates are likely to be FP detections of a mass. The final output of the integrated CAD scheme for the mass and polyp detection is obtained by combining the outputs of the quadratic classifiers from the analyses of the polyp and mass candidates.

The methods for the detection and extraction of the mass candidates involve assignment of several numerical parameter values. The assignment of these values is discussed briefly below.

Detection of Intraluminal Masses: Fuzzy Merging

As discussed above, the CAD polyp detector may generate several initial detections on the surface regions of large unobstructed intraluminal masses. Therefore, to reduce the computational burden introduced by mass detection, a separate detection step for intraluminal masses is not implemented, but they are identified by use of a new fuzzy merging method that analyzes the fuzzy memberships of the initial polyp detections. The detections that are determined to originate from the same lesion are considered as an initial mass detection and are processed separately from the polyp detections.

Let U be a universal set. A fuzzy set F in U can be defined as a set of ordered pairs as
F={(c,μ_{F}(c))|c∈U}, (10)
where μ_{F}(.) is the membership function (or characteristic function) of F, and μ_{F}(c) is the grade (or degree) of membership of c in F [58]. The membership function μ_{F}(.) maps U to the membership space M, where usually M=[0, 1]. In a crisp set, M={0,1}. An α-cut of a fuzzy set F is a crisp set F_{α} that contains all elements of the universal set U with a membership grade in F greater than or equal to α:
F_{α}={c∈U|μ_{F}(c)≧α}, α∈(0,1]. (11)

In our application, the input data are the set of 26-connected regions of the initial CAD polyp detections. The regions that are located beyond Dₘₐₓ=50 mm from each other may not be merged, and their membership grade is always zero. Otherwise, for two regions, A and B, within Dₘₐₓ, the symmetric membership function is defined by
μ_{A}(B)=μ_{B}(A)=(w_{CT}μ_{A,B}^{CT}+w_{GR}μ_{A,B}^{GR})ε_{D}(A,B), (12)
where the functions μ_{A,B}^{CT} and μ_{A,B}^{GR} characterize the membership between A and B based on the CT and gradient values along a 3-D sampling ray R between the centers of A and B. Here, the sampling ray R is defined as a line between the geometric center voxels of A and B. As described below, the value of μ_{A}(B) is defined to become high when regions A and B are on a single lesion. The weights w_{CT} and w_{GR} are generally set to (w_{CT},w_{GR})=(0.5,0.5), but may be modified in situations where it is obvious that A and B should not be merged. The function ε_{D}(A,B) decreases the membership grade as the distance between A and B increases, as discussed below.

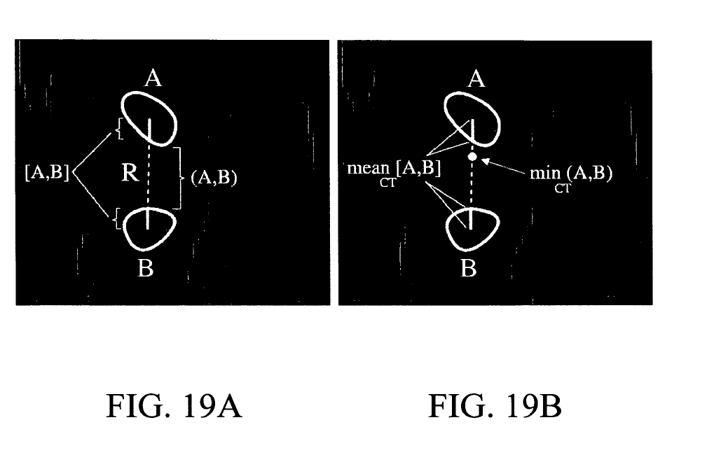

To calculate μ_{A,B}^{CT}, the minimum CT value along R between the regions of A and B, denoted by min_{CT}(A,B), is first computed (FIGS. 19A-19B). Next, the average CT value along R within the regions of A and B, or mean_{CT}[A,B] is determined. The ratio of these values, or,

$${f}_{A , B}^{CT} = \frac{{min}_{CT} ( A , B )}{{mean}_{CT} [ A , B ]} ,$$

is scaled linearly to [0,1] in order to characterize the membership of A and B based on the CT value:

$${μ}_{A , B}^{CT} = \{ \begin{matrix}1.0 & {f}_{A , B}^{CT} > 0.9 \\ 0.0 & {f}_{A , B}^{CT} < 0.5 \\ \frac{{f}_{A , B}^{CT} - 0.5}{0.9 - 0.5} & otherwise\end{matrix} \qquad ( 13 )$$

The value μ_{A,B}^{CT} is high if the CT values along R are relatively uniform, indicating that the detections appear within the same lesion. The value is low if the CT value is attenuated significantly between the regions, suggesting that the detections may not appear within the same lesion.

To calculate μ_{A,B}^{GR}, one determines the maximum gradient between the regions A and B, or max_{GR}(A, B), and divides it by the maximum value of the gradient within A and B, or max_{GR}[A,B]. The resulting value,

$${f}_{A , B}^{GR} = \frac{{max}_{GR} ( A , B )}{{max}_{GR} [ A , B ]} ,$$

is scaled as follows:

$${μ}_{A , B}^{GR} = \{ \begin{matrix}1.0 & {f}_{A , B}^{GR} < 1.1 \\ 0.0 & {f}_{A , B}^{GR} > 3.0 \\ \frac{{f}_{A , B}^{GR} - 1.1}{3.0 - 1.1} & otherwise\end{matrix} \qquad ( 14 )$$

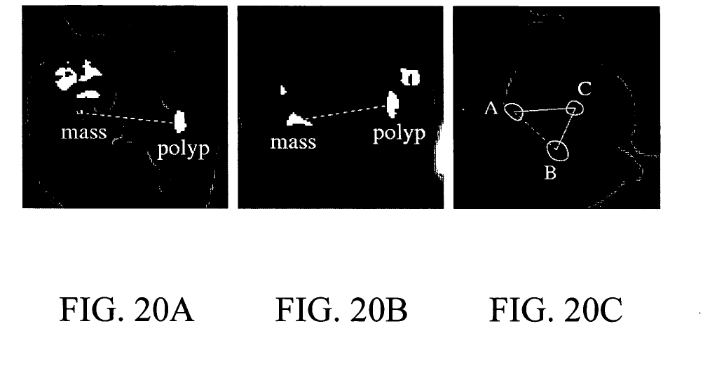

The value μ_{A,B}^{GR} is high if the highest gradient between A and B is low, i.e., there is no clear separating boundary between the regions (FIG. 20A). The value is low if the gradient is high, indicating that there is a boundary (or boundaries) between the regions. Such boundaries may be caused not only by the surface of the colonic wall, but also by a sharp change in the local tissue characteristics. In such cases, the regions A and B are likely to belong to different lesions.

The numerical boundary values in Eqs. (13) and (14) were determined experimentally, as discussed in Section 4. For example, in Eq. (13), the threshold value 0.9 implies that if the CT value is reduced less than 10% between A and B, the two regions A and B definitely belong to the same lesion. Similarly, if the CT value is reduced by more than 50% between A and B, the regions definitely do not belong to the same lesion.

If the CT value between A and B is less than −700 HU, it is likely that there is air between the regions and they do not represent the same lesion. However, the value of μ_{A}(B) may not always reflect this clearly if equal weights are used. Therefore, in this situation, the effect of the CT value is weighted by modifying the membership weights in Eq. (12) as follows:

$${w}_{CT} = 0.5 + 0.5 min \{ 1 , \frac{700 + {min}_{CT} ( A , B )}{- 324} \} , \\ {w}_{GR} = 1.0 - {w}_{CT} . \qquad ( 15 )$$

The membership grade between A and B is also dependent on their distance. The effect of ε_{D}(A,B) is to reduce the membership grade for detections that are far apart as compared with detections that are very close. As the distance D(A,B) between A and B increases, it becomes increasingly likely that they originate from a different lesion, unless the membership grade based on the CT value and gradient is very high (FIGS. 20A-20B). We calculate this effect by

$${ɛ}_{D} ( A , B ) = \{ \begin{matrix}1 & D ( A , B ) < 10 mm \\ 0 & D ( A , B ) > {D}_{max} \\ \frac{{D}_{max} - D ( A , B )}{{D}_{max} - 10} & otherwise\end{matrix} \qquad ( 14 )$$

Once the memberships have been established, the detected regions that have a high membership grade are merged by determining the α-cut of the regions. The threshold for this hard clustering is α=0.5, but the regions with the highest membership are merged first. If, during this merging process, one or both of the regions A and B to be merged already belongs to a previously merged region, e.g., A⊂E, where E is a merged region, it is checked that the distance between all components of E and B (or all components of B, if B is also a previously merged region) are within Dₘₐₓ. Because of this constraint, the merging process cannot spread over a long “chain” of candidates along the colonic wall. On the other hand, masses with complicated shapes can be detected, because a high membership grade is required only between two detections within a region (FIG. 20C).

The merging process ends when no mergeable regions can be found. The regions that were constructed by merging of initial detections are tested for their spatial spread along their three perpendicular principal axes for estimating the sizes of the lesions that the merged regions represent. A region is considered a mass candidate if the spread exceeds 25 mm along one of the principal axes, or if the largest of these spreads is 22.5-25 mm and the spreads in the two corresponding perpendicular directions are >50% and >75% from the largest spread. Small polyp-like lesions have a small maximum spread, or their spreads in the directions perpendicular to the maximum spread are small. The detections that are not labeled as mass candidates are considered polyp candidates and are processed as described in below.

Detection of Non-Intraluminal Masses: Wall-Thickening Analysis

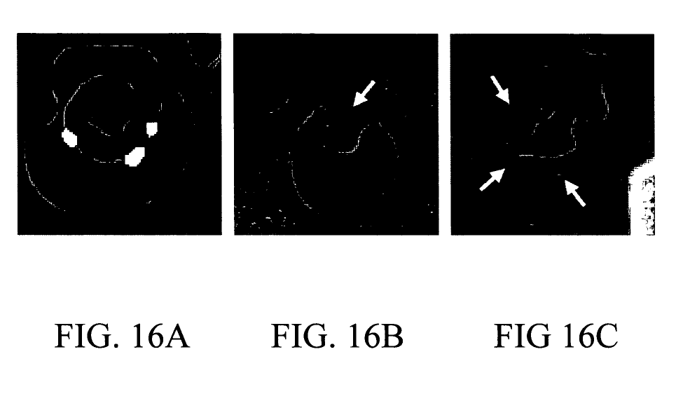

As shown in FIG. 16C, non-intraluminal masses are associated with a thickening of the colonic wall that can be seen as a thick region of locally high density surrounding the colonic lumen. The present method for detecting such regions, called wall-thickening analysis, is similar to the one suggested by Vining et al. [45] for polyp detection: at each surface voxel of the colonic wall, a sampling ray L is projected along the surface normal into the colonic wall, and estimate the wall thickness based on the CT values along the ray. However, compared with the method of Vining et al., the present method is more specific to the application of the detection of non-intraluminal masses and involves several additional steps.

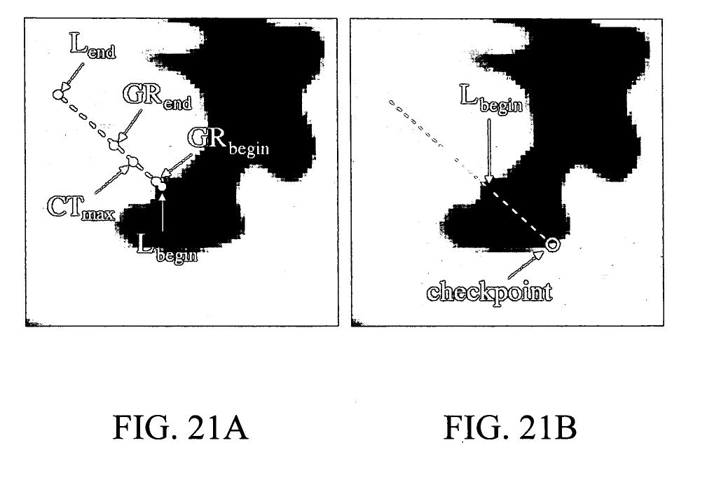

First, the CT values along the sampling ray L with a length of 20 mm are averaged by a 3-voxel window to reduce noise. Let the start and end locations of L be denoted as L_{begin} and L_{end} (FIG. 21A). To determine whether abnormal thickening is present, the location of the maximum CT value, CTₘₐₓ, between L_{begin} and L_{end} is first calculated. Next, the region of the colonic wall is determined by determining the locations of the gradient maxima, GR_{begin} and GR_{end}, between L_{begin} and CTₘₐₓ and between CTₘₐₓ and L_{end}, respectively. The region between GR_{begin} and GR_{end} is expected to represent the colonic wall, and is considered as being suspiciously thickened if the following six criteria hold:
1. To determine whether the region between GR_{begin} and GR_{end} has locally increased density, representing a thickened region of the colonic wall, one calculates the average CT value, CT_{wall}, between GR_{begin} and GR_{end}, and the average CT value, CTₜᵢₛₛᵤₑ, between GR_{end} and L_{end}. To detect a thickened region, we impose CT_{wall}>1.025×CTₜᵢₛₛᵤₑ.
2. Except for the lumen, the colonic wall should have a solid tissue structure. Therefore, the condition that CT>−200 HU between GR_{begin} and L_{end} is imposed.
3. The wall region should be clearly delineated: GR_{end}>10 HU is imposed.
4. To ensure that osseous structures, possibly interfering with the analysis, are not present, CTₘₐₓ<300 HU is imposed.
5. The distance between GR_{begin} and GR_{end} should be at least 5 mm for the wall to be considered as having potentially abnormal wall thickening.
6. To exclude situations where a high-density region caused by internal organs adhering to the colonic wall could be identified incorrectly as wall thickening, the condition that the distance between L_{begin} and GR_{begin} is at most 2.5 mm is imposed, and that the distance between GR_{end} and L_{end} is at least 3 mm.

Once the voxels representing potential regions of abnormal thickening have been detected, a symmetry check is performed by projecting an inverse sampling ray from the detected voxels through the colonic lumen to the opposite side of the colonic wall (FIG. 21B). The detected thickening at a voxel is not considered valid unless thickening is also detected on the opposite side of the wall. Thus, the method detects only a nearly circumferential type of wall thickening, as examples of a flat, non-circumferential abnormal wall thickening are not available in the database.

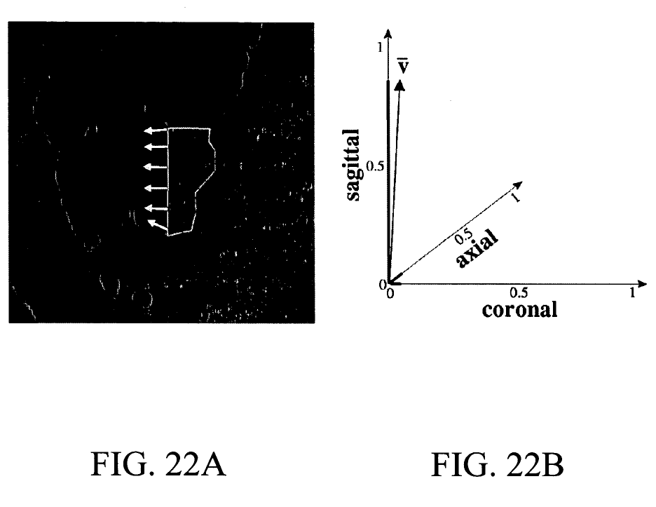

To reduce false positives, additional checks are performed for the extracted, suspicious regions. The total volume of the region should be at least 3000 mm³, and there should be at least 300 voxels associated with the locations where suspicious thickening was originally detected. Furthermore, to eliminate FP detections due to fluid, the directions of the gradient vectors within the suspicious region were analyzed. If more than 50% of the region is associated with unit vectors of gradient with a sagittal component ≧0.9 in magnitude, the region is considered to be fluid and is eliminated from further analysis (FIGS. 22A-22B). This check does not increase the computational complexity of the detection of masses, because the gradients have already been calculated to detect the regions associated with the wall thickening.

Extraction of the Mass Region by Use of Level Sets

The mass candidate regions, detected by the two methods described in the previous sections, are used as seeds for extracting the final mass regions. The extraction step is based on a level set method [59]. The basic idea of the level set method in extracting regions is to allow the initial surface γ of a smooth, closed object to move along its normal vector field with speed F. In this application, the initial regions of the mass candidates provide the initial conditions for the level set method. To extract the complete region of a mass candidate, solve the following flow evolution equation:
φ+(g_{I}+c_{I})(1−εκ_{M})|∇φ|−β∇P·∇φ=0, (17)
where φ is the level set function to be solved. The zero level set of φ represents the evolving front, or surface, of the extracted region. Ideally, the surface should pass the minor boundaries in the input data and settle to the major boundaries. The front can be considered to expand with a speed 1−εη_{M}, where η_{M} is the mean curvature of the surface, and ε controls the effect of the curvature. The additional terms in Eq. (8) represent expansion or contraction forces that control how the level set surface evolves.

The gradient-based expansion force g_{I} is

$${g}_{I} ( x , y , z ) = \frac{1}{1 + max \{ 0 , \vert ∇ ( {G}_{σ} * I ( x , y , z ) ) \vert - 10 \}} , \qquad ( 18 )$$

where G_{σ}*I denotes the CTC data set convolved with a 3-D Gaussian smoothing filter with a standard deviation σ=0.5. If there is no boundary gradient, the expansion force is g_{I}=1.

In addition to the gradient-based expansion force, we introduce an expansion force c_{I} based on the CT value (in HU):

$${c}_{I} ( x , y , z ) = \frac{1}{1 + max \{ 0 , ( \vert {G}_{σ} I ( x , y , z ) \vert + 700 ) / 100 \}} . \qquad ( 19 )$$

In the present invention, the use of c_{I} is necessary because the automatically detected initial regions may be located at both sides of the mass boundary. Therefore, without c_{I}, the evolving level set front could spread into the colonic lumen.

The surface tension force, |∇φ|, controls the smoothness of the evolving level set surface, and depends on the mean curvature which may be calculated from the level set function φ as

$${κ}_{M} = ∇ \times \frac{∇ ϕ}{\vert ∇ ϕ \vert} = \frac{\begin{matrix}( {ϕ}_{yy} + {ϕ}_{zz} ) {ϕ}_{x}^{2} + ( {ϕ}_{xx} + {ϕ}_{zz} ) {ϕ}_{y}^{2} + \\ ( {ϕ}_{xx} + {ϕ}_{yy} ) {ϕ}_{z}^{2} - 2 {ϕ}_{x} {ϕ}_{y} {ϕ}_{xy} - \\ 2 {ϕ}_{x} {ϕ}_{z} {ϕ}_{xz} - 2 {ϕ}_{y} {ϕ}_{z} {ϕ}_{yz}\end{matrix}}{{( {ϕ}_{x}^{2} + {ϕ}_{y}^{2} + {ϕ}_{z}^{2} )}^{3 / 2}} . \qquad ( 20 )$$

The force that attracts the evolving surface toward a boundary is implemented as the gradient of a potential field defined by
P(x,y,z)=−(max{0,|∇(G_{σ}*I(x,y,z))|−20}). (21)
The parameter β in Eq. (15) controls the effect of the force that attracts the evolving surface to a boundary.

The flow evolution of Eq. (15) was implemented similarly to the two-stage approach described by Sethian et al. [59]. First, the flow evolution was solved by use of the expansion forces and a fast marching method. The fast marching method was realized by solving of the Eikonal equation
|∇T|g_{I}=1,Γ={(x,y)|T(x,y)=0}, (22)
where Γ is the initial position of the level set surface. The iteration continues until the solution |∇T| becomes large and, hence, the flow has almost stopped because it reached the desired boundary. To implement the fast marching method, write the Eikonal equation as

$${\{ {max ( {D}_{ijk}^{- x} T , - {D}_{ijk}^{+ x} T , 0 )}^{2} + {max ( {D}_{ijk}^{- y} T , - {D}_{ijk}^{+ y} T , 0 )}^{2} + {max ( {D}_{ijk}^{- z} T , - {D}_{ijk}^{+ z} T , 0 )}^{2} \}}^{1 / 2} = \frac{1}{{g}_{I}} , \qquad ( 23 )$$

where the terms DᵢⱼₖT represent approximate forward and backward operators on T. For example,

$${D}_{ijk}^{+ x} T = \frac{T ( i + h , j , k ) - T ( i , j , k )}{h} and$$

$${D}_{ijk}^{- y} T = \frac{T ( i , j , k ) - T ( i , j - h , k )}{h} .$$

The fast marching method can be implemented efficiently by observing that the upwind difference structure of Eq. (23) allows propagation from small to large values of T. Only the values around the thin evolving surface boundary need to be considered, and these can be updated by use of a min-heap data structure. The details of the implementation are discussed in Sethian et al. [59].

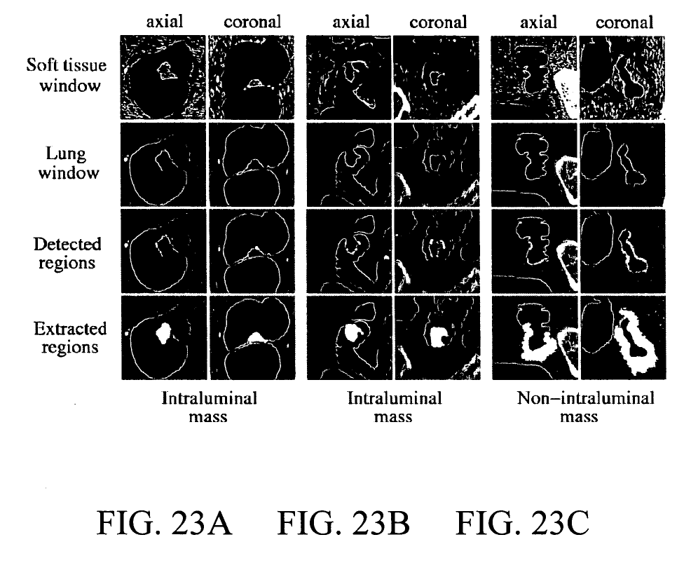

Once the final level set surface has been approximated by the fast marching method, a more precise, narrow band level set method could be used for obtaining the final solution [59]. However, in this application, the fast marching method was applied a second time by including the boundary-attracting force in the second term of Eq. (15). Three examples of the final extracted regions of masses are shown in FIGS. 23A-23C. As shown in the bottom figures, the coverage of a mass by the extracted region is not necessarily precise, but most of the mass region is covered.

Selection of the Parameter Values and Features

Most of the numerical parameter values of the methods of mass detection and extraction were determined empirically. For the fuzzy merging method, the numerical threshold values in Eqs. (13)-(15) were determined by examination of the CT and gradient values between the mass and polyp CAD detections in one data set that included five polyps and a circumferential mass. For the wall-thickening analysis, most of the parameter values were estimated visually from one dataset containing a typical wall-thickening type of mass. The requirements that a detected region should have a minimum volume of 3000 mm 3 and be associated with ≧300 voxels indicating wall thickening were established by first calculating these values for both non-intraluminal masses: the corresponding minimum values for all data sets were approximately 3600 mm³ and 380 voxels, respectively.

The values of the parameters ε=0.4 and β=0.66 of the level set method, as well as the threshold values in Eqs. (16), (17), and (19), were adjusted approximately by a visual examination of the resulting extracted mass regions.

A combination of two features was used to reduce FP mass detections by use of a quadratic classifier. The use of more than two features would improve the performance of the mass detection, but would also reduce the generalizability of the results. Because only fourteen masses were used in this study, the use of additional features might cause the classifier to over learn the data. With only two features, it is likely that the results obtained in this study can be reproduced when a larger database is used for the evaluation of our mass detection methods.

The features were chosen by evaluation of the FP reduction for combinations of the mean, variance, and the mean of the 10 maximum values of the CT value, the shape index, and the gradient within the regions of the detected mass candidates. The calculation of these features had been implemented previously in the CAD scheme for the detection of polyps. The features were designed to characterize the shape or internal texture of the detected regions for an effective reduction of FPs.

Performance Evaluation Method

The detection results were characterized primarily with a by-mass analysis, where a mass is considered detected if it is detected in either or both supine and prone positions of the patient. Because each abnormal patient had only one mass, the results of the by-mass and by-patient analysis were identical. In a by-patient analysis, an abnormal patient is considered detected if at least one mass is detected in the supine or prone view of the patient. Furthermore, we calculated the detection sensitivities when only prone data sets or only supine data sets were used in the evaluation.

The performance of the detection of intraluminal masses was evaluated by use of the leave-one-out (round-robin) method, which is expected to provide a good estimate of the unbiased performance of a classifier when the available data are limited. The leave-one-out evaluation was implemented with a by-patient elimination. That is, for a given patient, all candidates detected in the patient were removed from the set of all candidates, the quadratic classifier was trained with the remaining candidates, and the resulting discrimination function was tested on the candidates that were removed. This has the effect of reducing the potential bias caused by the use of the data from the same patient for training and testing. Once this process had been repeated for each patient, the discrimination function was thresholded at various detection sensitivity levels to generate a free-response operating characteristic (FROC) curve [61].

Because of the small number (2) of non-intraluminal masses in our database, it was not feasible to perform a leave-one-out evaluation for the wall-thickening analysis. Therefore, for this method, the quadratic classifier was trained with all detections of the non-intraluminal mass detection method and evaluated the result.

The mass detection performance of the CAD scheme was also investigated for the detection of polyps without the use of the explicit mass detection methods. That is, the inventors investigated how many masses would have been detected if the CAD scheme for polyp detection had been used. The quadratic classifier of the CAD scheme for polyp detection was trained and tested with the 82 cases and a leave-one-out method by treating the polyps (5-25 mm) as TPs and other detections as FPs. Then, the number of masses (>25 mm) that were detected as polyps by this CAD scheme at each detection sensitivity level was determined.

We retrospectively collected 82 cases (patients) from standard CTC examinations performed at the University of Chicago Hospitals (80 cases) and the Beth Israel Deaconess Medical Center (2 mass cases). In preparation for same-day optical colonoscopy, all patients underwent a colon-cleansing regimen with polyethylene glycol. The CTC scanning was performed with helical single- and multi-slice CTC scanners (HiSpeed CTi or LightSpeed QX/i, General Electric Medical Systems, Milwaukee, Wis.) in supine and prone positions with reconstruction intervals of 1.0-5.0 mm (within 1.5-2.5 mm: 91%), slice thicknesses of 1.25-5.0 mm (within 5.0 mm: 80%; within 2.5 mm: 15%), tube currents of 50-160 mA (within 100 mA: 87%) with 120 kVp, and tube rotation times of 1.0 (single scan) and 0.8 (multi-scan) seconds. The CTC data sets represented the entire region of the colon, from the rectum to the lung diaphragm. To yield 3-D isotropic data sets, the original 512×512 CT images were interpolated linearly in the axial direction to a voxel resolution of 0.51-96 mm (average: 0.69 mm).

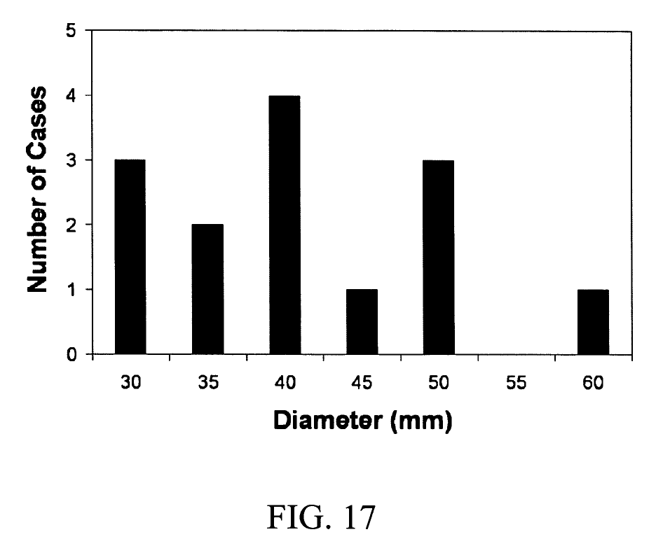

There were 14 colonoscopy-confirmed masses (12 intraluminal, 2 non-intraluminal) measuring approximately 30-60 mm in diameter (FIG. 17) in 17% of the cases (Table V). Seven of the intraluminal masses were polypoid or lobular, three were annular or circumferential, and two were semi-circumferential. The diameter of the masses was calculated by use of colonoscopy reports and by measurement of the average diameter of the mass in the reconstructed CT volume. For the two non-intraluminal masses, the mass diameter was calculated as the maximum distance between the centers of the thickened wall on opposite sides of the colonic lumen. The colonoscopic examination was incomplete in some of the mass cases, but no polyps nor additional masses could be found by an experienced radiologist in the CTC data in such cases. One of the masses, located at the rectum, was only partially visible in both supine and prone positions, because it was cut off by the CT reconstruction cylinder at the time when the CT scan was performed.

There were 30 colonoscopy-confirmed polyps measuring 5-25 mm in 19% of the cases. Four cases had both polyps and masses, resulting in a total of 15 polyps and 4 masses. Fifty-six cases (68%) were normal, i.e., a complete optical colonoscopic examination did not detect any colorectal neoplasms in these cases.

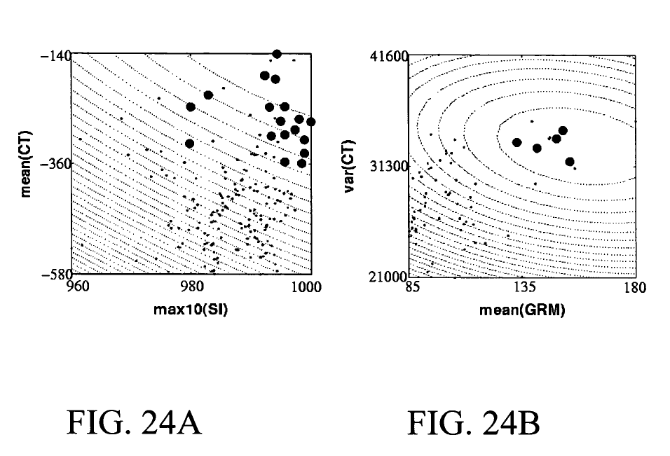

The fuzzy merging method detected all but one of the intraluminal masses, or 79% of all masses. After the initial detection step, there were 165 FP initial mass candidates. The two features used to reduce FPs are shown in FIG. 24A. At the 79% by-mass detection sensitivity level, the average FP rate based on the leave-one-out evaluation was 0.16 FPs per patient.

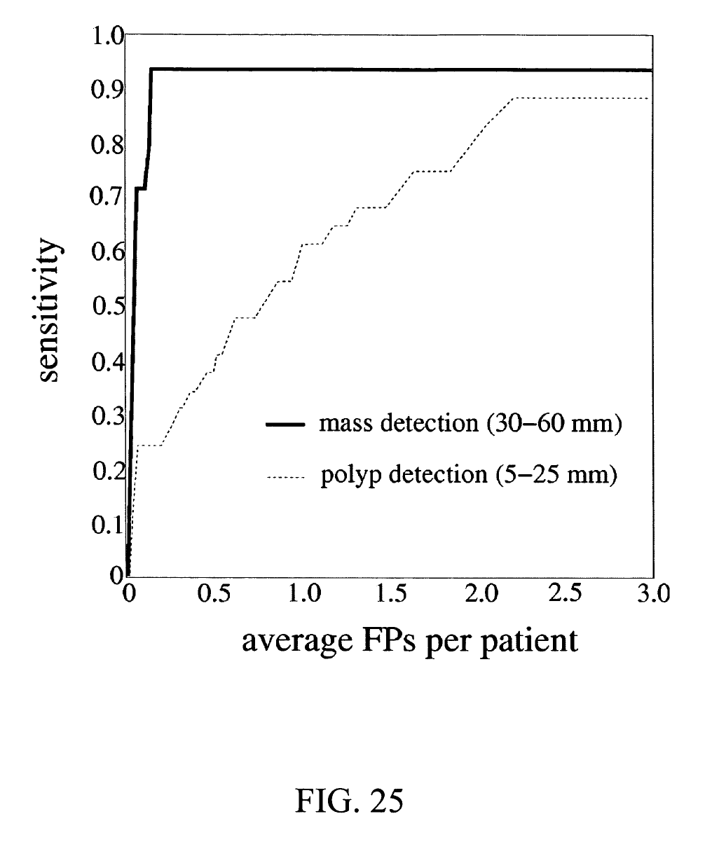

The wall-thickening analysis detected both of the non-intraluminal types of masses in the database, as well as one of the intraluminal circumferential masses with apple-core morphology. The latter mass resembled the wall-thickening type of non-intraluminal mass, since it obstructed the colon circumferentially. There were 69 FP initial mass candidates, but most of these could be eliminated by use of the quadratic classifier with two features (FIG. 24B). The resulting FP rate was 0.05 FPs per patient on average. To characterize the overall performance of the combination of the fuzzy merging and wall-thickening analysis, we generated a FROC curve (FIG. 25) by adding the sensitivity and FP-rate of the wall-thickening analysis to the FROC curve obtained with the fuzzy merging method. The combined performance showed approximately 93% by-mass detection sensitivity (13 out of the 14 masses were detected) with an average FP rate of approximately 0.21 FPs per patient.

The FROC curve obtained in this manner is expected to represent the combined mass detection performance well, because the two mass detection methods run independently and the number of false positives generated by the wall-thickening analysis is negligible. When only prone data sets of the patients were used in the mass detection, the fuzzy merging method detected 10 of the 12 (83%) intraluminal masses, and the wall-thickening method detected 2 of the 2 (100%) non-intraluminal masses. In combination, the two methods detected 12 of the 14 (86%) masses in prone data sets with 0.10 FPs per patient on average. When only supine data sets were used, the fuzzy merging method detected 8 of the 12 (67%) intraluminal masses, and the wall-thickening analysis detected 100% of the non-intraluminal masses. In combination, the methods detected 10 of the 14 (71%) masses in supine data sets with 0.61 FPs per patient on average.

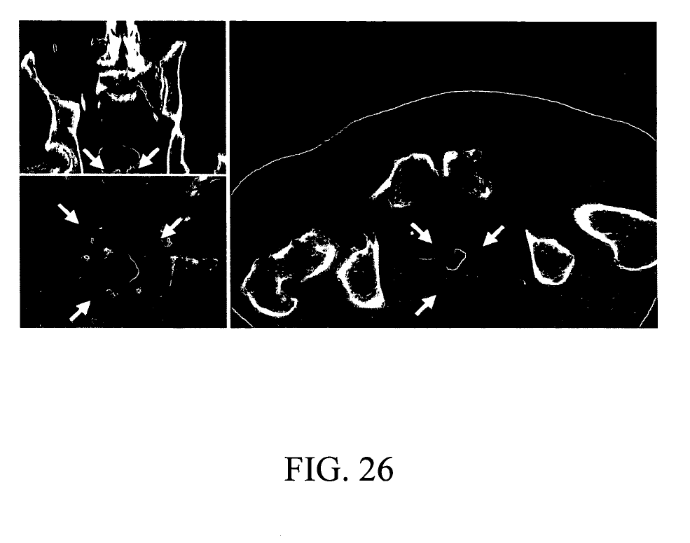

The mass that was missed by both mass detection methods was located at the rectum and was partially cut off from the CTC data set in both supine and prone positions (FIG. 26). The mass can be seen only partially in the last five CT images of the original data. The fuzzy merging method failed to identify the mass candidate because the initial detections on the mass were located within a short distance and only on one side of the lesion. The wall-thickening analysis failed to identify the mass because the region of the mass was too thin to be recognized as a wall thickening.

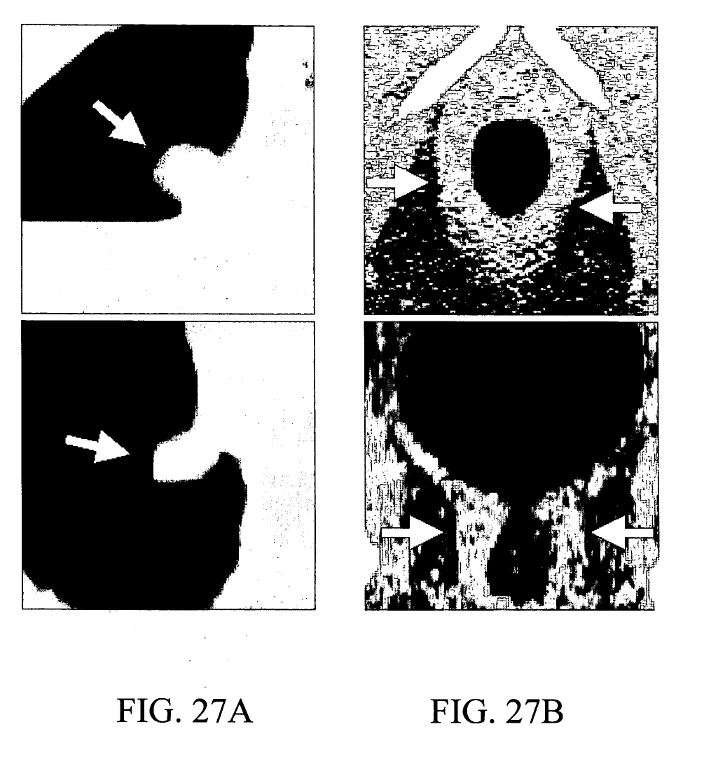

To verify that the mass detection methods work consistently, and to identify potential problems, we also analyzed the sources of the FP detections produced by both mass detection methods. The FP polyp detections generated by the CAD method for the detection of polyps have been analyzed similarly in previous studies [49,62] by the present inventors. The mass detection methods generated only few false positives. The fuzzy merging method generated 13 FP mass detections for the 164 data sets. There was no particular single source of FPs, but 43% of the FPs were associated with a rectal wall with bump-like structures caused by a combination of residual stool, hemorrhoids, and extrinsic compression by the rectal tube. Other sources included a dilated fold with stool or a dilated ileocecal valve (31%) (FIG. 27A). The wall-thickening analysis generated only 4 FP detections, and these were all associated with normal circumferential thickening at the rectum (FIG. 27B).

Finally, we evaluated the mass detection performance of the CAD scheme developed for the detection of polyps alone, as described below. At a 90% by-polyp detection sensitivity level, only two of the masses (14%) were among the final output of the scheme. At a 97% by-polyp detection sensitivity level, five (36%) of the masses were among the polyp detections. Therefore, the CAD scheme for polyp detection cannot be considered effective for the detection of large masses.

Both mass detection methods missed a mass that was partially cut off from the CTC data in both supine and prone positions. This suggests that a specialized method may need to be developed for checking for abnormalities within the boundary regions of the CT data reconstructed from axial images. On the other hand, both methods were able to detect masses that were only partially visible due to a colonic segment collapsing on the mass. In these cases, the complete region of the mass had been imaged by the CTC scanner, and, thus, an analysis of the internal structure of the mass was performed, leading to the detection of the mass.

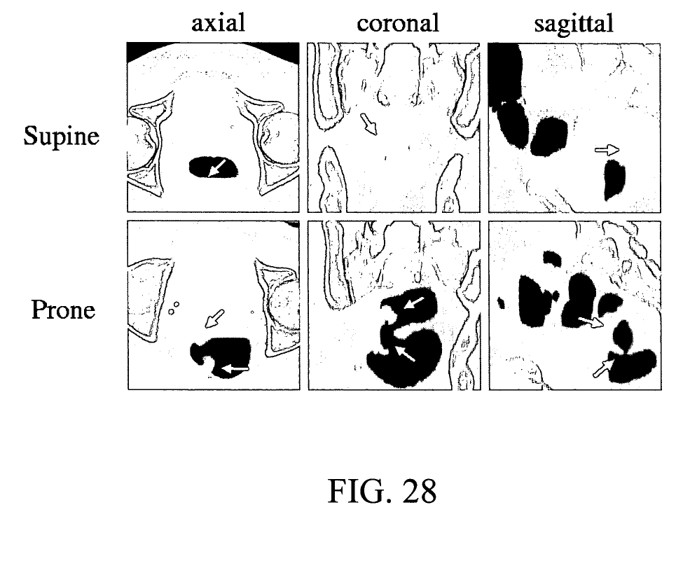

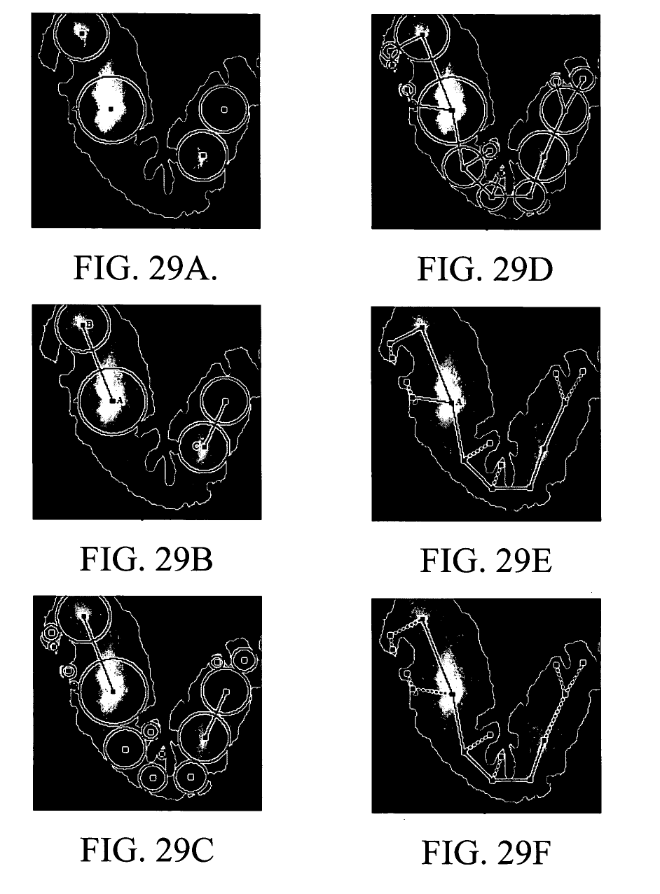

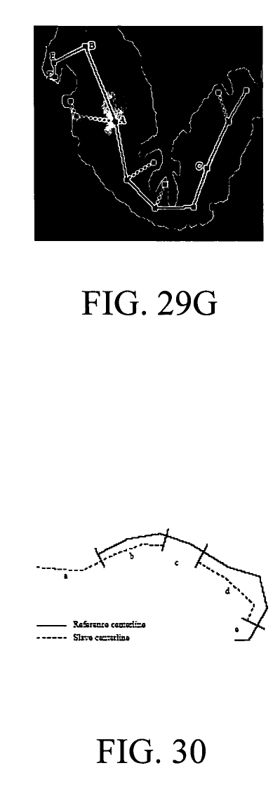

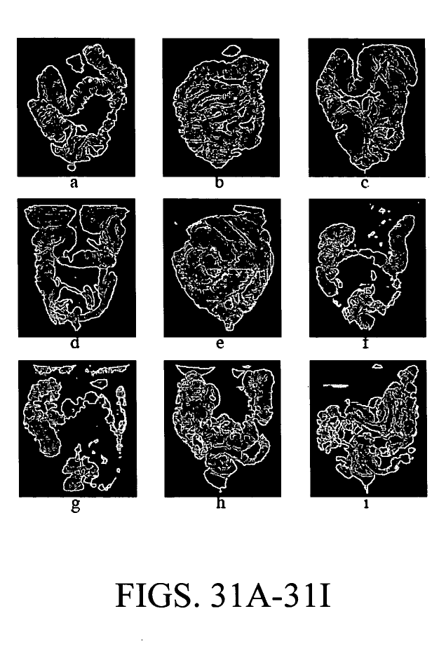

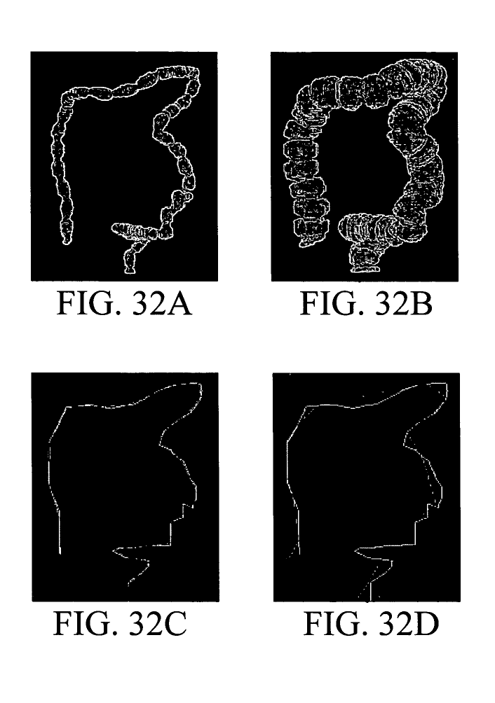

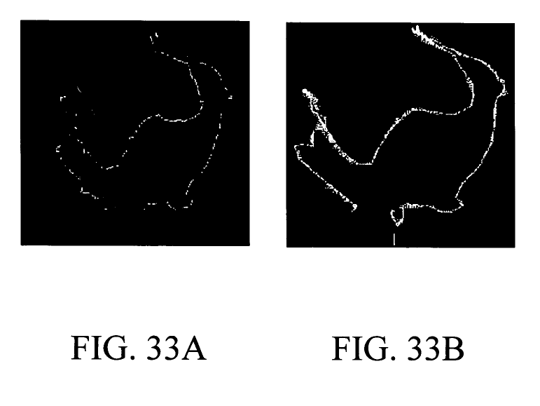

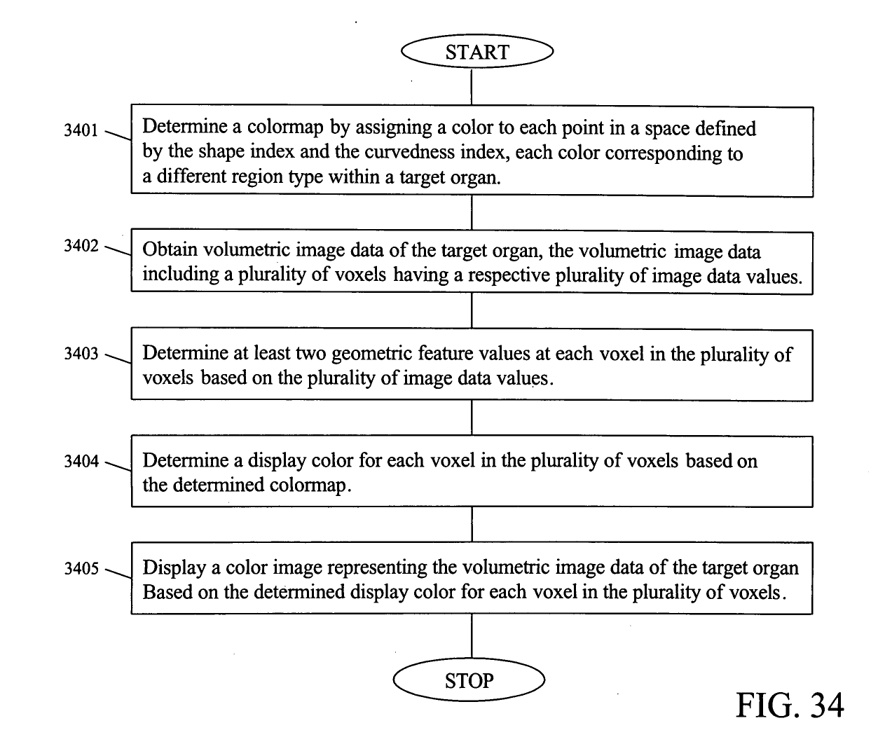

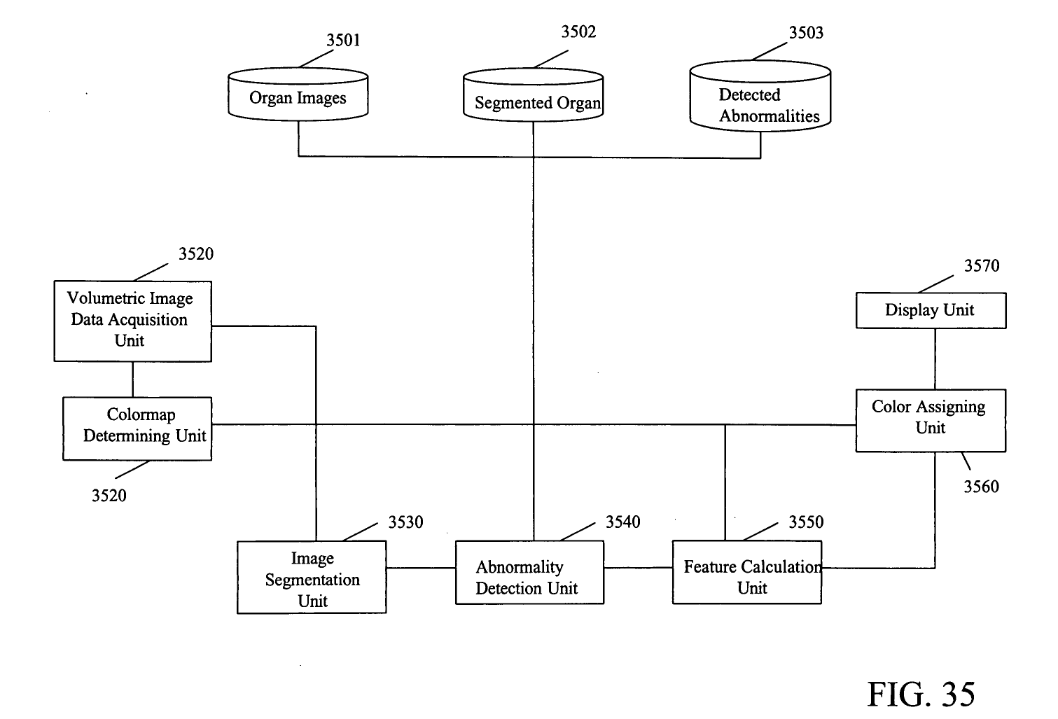

In the case of intraluminal masses, the highest detection performance was obtained when both supine and prone data sets were used. This is because some masses that were clearly visible in one position of the patient were invisible in the other position due to a collapsed region or residual fluid, as shown in FIG. 28. In particular, in this study, the performance of the mass detection was higher in the prone data sets than in the supine data sets. This imbalance can be explained by the locations of the masses: 75% of the intraluminal masses were located within the ascending colon, the descending colon, or the rectum. Because of the anatomy of the colon, these colon segments tend to have a better distension in the prone position than in the supine position. Therefore, the use of only supine data sets provided the lowest detection performance in this study.

The number of masses used in the evaluation was relatively small (14 masses in 14 patients). In particular, most of the masses were of the intraluminal type, and only two of the masses were of the non-intraluminal type which was detected by an analysis of the thickness of the colonic wall. Therefore, even though the evaluation was based on a leave-one-out evaluation with a large number (164) of CTC data sets, it is premature to make precise estimates about the performance of our CAD scheme when it is used with a larger population. Nevertheless, it appears that the detection of masses does not affect the result of the polyp detection, and each type of lesion was identified correctly and processed separately by the corresponding method. In particular, each detected polyp was identified correctly as a polyp, and each detected mass was identified correctly as a mass. The initial results suggest that a high sensitivity and a low FP rate may be obtained with automated detection of masses.

The leave-one-out evaluation was performed by use of by-patient elimination, where all detections of a patient are removed at each evaluation step, one patient at a time. The results are expected to estimate the performance of the CAD scheme in a situation where an unseen case is presented to the scheme. On the other hand, because of the relatively small number of masses and polyps, some of the CTC cases are unique in the sense that similar lesions are not found in the other cases. Therefore, the leave-one-out performance with by-patient elimination might give an overly conservative estimate of the performance of the CAD scheme.

Although the current implementation of the CAD scheme has not been optimized for speed and efficiency, it is relatively fast. The complete process of detecting polyps and masses starting from the original CT images in DICOM format and including disk access takes approximately 10 minutes when a PC workstation (Marquis K120-SR, ASL Inc., Newark, Calif.) with dual processors (Athlon MP 1.2 GHz, AMD, Sunnyvale, Calif.) is used. The detection of intraluminal masses by the fuzzy merging method takes only a few seconds, the detection of non-intraluminal lesions takes about 30 seconds, and the level set extraction takes about 30 seconds per mass candidate.

Two methods for the detection of colorectal masses were developed. The methods can be integrated into a CAD scheme for the detection of polyps for computational efficiency. All masses but one that was partially cut off from the CTC data sets were detected at a 93% sensitivity with an average FP rate of 0.21 detections per patient. No polyp was missed due to mass detection when a polyp detection scheme was combined with the mass detection. Integration of the computerized polyp and mass detection in a single CAD scheme appears to be a feasible approach to provide a comprehensive high-performance computer aid for radiologists in a clinical CTC screening setting.

The present method can also be implemented more generally on other medical images of other organs (e.g., mammographic breast images, or CT scans of the thorax, abdomen, or skeletal system) with respect to some other disease state or state of risk. Nodule or lesion feature values can readily be obtained from other medical images by those of ordinary skill in the art. For example, the detection of nodule or lesion feature values in various medical images is also well known in this art. See, e.g., U.S. Pat. No. 5,881,124 (Giger et al., Automated method and system for the detection of lesions in medical computed tomographic scans), the contents of which are incorporated herein by reference.

For the purposes of this description an image is defined to be a representation of a physical scene, in which the image has been generated by some imaging technology: examples of imaging technology could include television or CCD cameras or X-ray, sonar, or ultrasound imaging devices. The initial medium on which an image is recorded could be an electronic solid-state device, a photographic film, or some other device such as a photostimulable phosphor. That recorded image could then be converted into digital form by a combination of electronic (as in the case of a CCD signal) or mechanical/optical means (as in the case of digitizing a photographic film or digitizing the data from a photostimulable phosphor). The number of dimensions that an image could have could be one (e.g. acoustic signals), two (e.g. X-ray radiological images), or more (e.g. nuclear magnetic resonance images).

All embodiments of the present invention conveniently may be implemented using a conventional general purpose computer or micro-processor programmed according to the teachings of the present invention, as will be apparent to those skilled in the computer art. Appropriate software may readily be prepared by programmers of ordinary skill based on the teachings of the present disclosure, as will be apparent to those skilled in the software art.

As disclosed in cross-referenced U.S. patent application Ser. No. 09/773,636, a computer 900 may implement the methods of the present invention, wherein the computer housing houses a motherboard which contains a CPU, memory (e.g., DRAM, ROM, EPROM, EEPROM, SRAM, SDRAM, and Flash RAM), and other optional special purpose logic devices (e.g., ASICS) or configurable logic devices (e.g., GAL and reprogrammable FPGA). The computer also includes plural input devices, (e.g., keyboard and mouse), and a display card for controlling a monitor. Additionally, the computer may include a floppy disk drive; other removable media devices (e.g. compact disc, tape, and removable magneto-optical media); and a hard disk or other fixed high density media drives, connected using an appropriate device bus (e.g., a SCSI bus, an Enhanced IDE bus, or an Ultra DMA bus). The computer may also include a compact disc reader, a compact disc reader/writer unit, or a compact disc jukebox, which may be connected to the same device bus or to another device bus.

Examples of computer readable media associated with the present invention include compact discs, hard disks, floppy disks, tape, magneto-optical disks, PROMs (e.g., EPROM, EEPROM, Flash EPROM), DRAM, SRAM, SDRAM, etc. Stored on any one or on a combination of these computer readable media, the present invention includes software for controlling both the hardware of the computer and for enabling the computer to interact with a human user. Such software may include, but is not limited to, device drivers, operating systems and user applications, such as development tools. Computer program products of the present invention include any computer readable medium which stores computer program instructions (e.g., computer code devices) which when executed by a computer causes the computer to perform the method of the present invention. The computer code devices of the present invention may be any interpretable or executable code mechanism, including but not limited to, scripts, interpreters, dynamic link libraries, Java classes, and complete executable programs. Moreover, parts of the processing of the present invention may be distributed (e.g., between (1) multiple CPUs or (2) at least one CPU and at least one configurable logic device) for better performance, reliability, and/or cost. For example, an outline or image may be selected on a first computer and sent to a second computer for remote diagnosis.

The invention may also be implemented by the preparation of application specific integrated circuits or by interconnecting an appropriate network of conventional component circuits, as will be readily apparent to those skilled in the art.

Moreover, parts of the processing of the present invention may be distributed for better performance, reliability, and/or cost. For example, an outline or image may be selected on a first computer and sent to a second computer for remote diagnosis.

Moreover, the invention provides computer program products storing program instructions for execution on a computer system, which when executed by the computer system, cause the computer system to perform the methods described herein.

Numerous modifications and variations of the present invention are possible in light of the above teachings. It is therefore to be understood that within the scope of the appended claims, the invention may be practiced otherwise than as specifically described herein.

TABLE I
Table I: Effect of colon phantom radius for centerline computation
efficiency. Kernels scale parameters used by the centerline algorithm
were set to 1.5 for seed points (seedscalefactor) and 1.1 for
regular points (regularscalefactor). Computer used for the experiment
was an Intel Pentium-based 800 MHz PC.

| | Phantom radius (voxels) | | | |
|---|---|---|---|---|
| | 3 | 10 | 20 | 30 |
| Number of maxima in DT | 2952 | 5587 | 11598 | 21838 |
| Number of maxima used | 60 | 59 | 56 | 95 |
| Distance map computation time (s) | 2.0 | 2.3 | 2.9 | 3.7 |
| Centerline computation time (s) | 2.4 | 2.4 | 2.7 | 2.9 |
| Mean displacement (mm) | 1.2 | 2.4 | 3.2 | 6.6 |
| Extension (%) | 94 | 100 | 100 | 93 |
| Max. displacement (mm) | 6.5 | 9.7 | 15.7 | 21.1 |
| Standard deviation | 1.0 | 2.3 | 3.2 | 5.8 |

TABLE II
Table II: Effect of different kernel scale parameters for centerline
computation efficiency. Colon phantom radius was fixed to 15 voxels.
Kernel size for regular points (regularscalefactor) was set to
70% of the seed point kernel scale factor (seedscalefactor). Computer
used was an Intel Pentium-based 800 MHz PC.

| | Seed point kernel scale factor | | | | |
|---|---|---|---|---|---|
| | 0.1 | 0.5 | 1.0 | 1.5 | 2.0 |
| Number of maxima | 8275 | 8275 | 8275 | 8275 | 8275 |
| in DT | | | | | |
| Number of maxima | 8200 | 1441 | 283 | 56 | 38 |
| used | | | | | |
| Centerline compu- | 20.2 | 2.7 | 2.4 | 2.6 | 2.6 |
| tation time (s) | | | | | |
| Mean displacement | 2.7 | 3.0 | 3.0 | 2.9 | 3.1 |
| (mm) | | | | | |
| Extension (%) | 100 | 100 | 100 | 100 | 100 |
| Max. displacement | 21 | 21 | 21 | 21 | 14 |
| (mm) | | | | | |
| Standard deviation | 3.5 | 3.7 | 3.7 | 3.5 | 2.5 |

TABLE III
Table III: Average differences between the centerlines.

| | | Mean Dis- | Exten- | Maximum | |
|---|---|---|---|---|---|
| Refer- | | placement | sion | Displacement | Standard |
| ence | Slave | (mm) | (%) | (mm) | deviation |
| A | Computer | 3.5 | 93 | 17 | 2.7 |
| B | Computer | 4.0 | 93 | 18 | 2.9 |
| C | Computer | 4.0 | 89 | 19 | 3.2 |
| A | B | 4.0 | 98 | 17 | 2.7 |
| B | A | 3.8 | 97 | 16 | 2.6 |
| C | A | 3.8 | 93 | 18 | 2.9 |
| A | C | 4.0 | 91 | 18 | 3.2 |
| B | C | 4.3 | 93 | 19 | 3.1 |
| C | B | 4.2 | 94 | 18 | 2.9 |

A, B, C = human-generated centerlines.
Computer = computer-generated centerline.

TABLE IV
Table IV: Average differences between the centerlines
when data were split into three categories -straightforward,
moderate, and challenging cases.

| | | Mean | Exten- | Maximum | |
|---|---|---|---|---|---|
| Refer- | | displacement | sion | displacement | Standard |
| ence | Slave | (mm) | (%) | (mm) | deviation |
| | | Straightforward | | | |
| A | Computer | 3.7 | 97 | 17 | 2.6 |
| B | Computer | 4.3 | 97 | 18 | 2.9 |
| C | Computer | 4.3 | 97 | 19 | 3.0 |
| A | B | 4.2 | 99 | 17 | 2.7 |
| B | A | 4.0 | 99 | 16 | 2.5 |
| C | A | 3.9 | 99 | 18 | 2.6 |
| A | C | 4.2 | 97 | 18 | 3.0 |
| B | C | 4.5 | 96 | 19 | 3.0 |
| C | B | 4.4 | 98 | 19 | 2.9 |
| | | Moderate | | | |
| A | Computer | 3.4 | 92 | 16 | 2.6 |
| B | Computer | 3.7 | 93 | 16 | 2.6 |
| C | Computer | 3.7 | 89 | 18 | 2.8 |
| A | B | 3.9 | 99 | 15 | 2.6 |
| B | A | 3.8 | 99 | 16 | 2.5 |
| C | A | 3.7 | 96 | 18 | 2.6 |
| A | C | 4.1 | 96 | 18 | 3.0 |
| B | C | 4.2 | 96 | 18 | 2.9 |
| C | B | 3.9 | 95 | 17 | 2.6 |
| | | Challenging | | | |
| A | Computer | 3.4 | 86 | 17 | 2.8 |
| B | Computer | 3.6 | 84 | 17 | 3.0 |
| C | Computer | 3.6 | 74 | 19 | 3.9 |
| A | B | 3.7 | 95 | 17 | 2.8 |
| B | A | 3.5 | 91 | 17 | 2.7 |
| C | A | 3.7 | 78 | 19 | 3.6 |
| A | C | 3.8 | 77 | 18 | 4.0 |
| B | C | 3.9 | 81 | 19 | 3.6 |
| C | B | 3.9 | 82 | 19 | 3.4 |

A, B, C = human-generated centerlines.
Computer = computer-generated centerline.

TABLE V
Distribution of the 14 masses and
30 polyps among the 82 CTC cases.

| | cases | masses | polyps |
|---|---|---|---|
| masses only | 10 | 10 | 0 |
| masses + polyps | 4 | 4 | 15 |
| polyps only | 12 | 0 | 15 |
| normal | 56 | 0 | 0 |
| total | 82 | 14 | 30 |

1. A method of displaying volumetric image data of a target organ as a visual aid in detecting at least one abnormality in the target organ, comprising:
obtaining the volumetric image data of the target organ, the volumetric image data including a plurality of voxels having a respective plurality of image data values;
determining at least two geometric feature values at each voxel in the plurality of voxels based on the plurality of image data values;
determining a colormap by assigning a color to each possible combination of the at least two geometric feature values, wherein each assigned color corresponds to a different region type within the target organ;
determining a display color for each voxel in the plurality of voxels based on the determined colormap; and
displaying, based on the determined display color for each voxel in the plurality of voxels, a color image representing the volumetric image data of the target organ.

2. The method of claim 1, further comprising:
identifying the at least one abnormality based on the displayed color image.

3. The method of claim 1, wherein the step of determining the colormap comprises:
selecting a region type of the target organ;
determining combinations of the at least two geometric feature values that most frequently correspond to the selected region type; and
assigning a color to each of the determined combinations of the at least two geometric feature values that correspond to the selected region type.

4. The method of claim 3, wherein the step of determining the colormap comprises:
obtaining simulated volume data representing the target organ; and
determining, based on the simulated volume data, the at least two geometric feature values for each voxel in the simulated volume data.

5. The method of claim 3, wherein the step of determining the colormap comprises:
obtaining clinical volume data representing the target organ; and
determining, based on the clinical volume data, the at least two geometric feature values for each voxel in the clinical volume data.

6. The method of claim 3, further comprising:
repeating the steps of selecting a region type, determining combinations, and assigning the color for a plurality of region types of the target organ; and
assigning a color for each possible combination of the at least two geometric feature values not associated with the selected region types by interpolating the colors assigned to the selected region types.

7. The method of claim 1, wherein the step of determining the at least two geometric features comprises:
determining, based on the plurality of image data values, a first index at each voxel, the first index being indicative of a local shape at each respective voxel; and
determining, based on the plurality of image data values, a second index at each voxel, the second index being indicative of a local scale at each respective voxel.

8. The method of claim 7, wherein the step of determining the first index comprises:
determining a shape index at each voxel, the shape index being indicative of a local topology at each respective voxel.

9. The method of claim 7, wherein the step of determining the second index comprises:
determining a curvedness index at each voxel, the curvedness index being indicative of a magnitude of a local curvature at each respective voxel.

10. The method of claim 1, further comprising:
detecting an abnormality region in the target organ based on calculated feature values at each voxel in the plurality of voxels,
wherein the displaying step further comprises displaying the detected abnormality region in a predetermined color.

11. The method of claim 1, wherein the obtaining step comprises:
obtaining a set of cross-sectional images of the target organ;
obtaining a set of voxels representing a total scanned volume from the set of cross sectional images; and
performing segmentation to extract, from the set of voxels representing the total scanned volume, the plurality of voxels in the volumetric image data of the target organ.

12. A system for displaying volumetric image data of a target organ as a visual aid in detecting at least one abnormality in the target organ, comprising:
a mechanism for obtaining the volumetric image data of the target organ, the volumetric image data including a plurality of voxels having a respective plurality of image data values;
a mechanism for determining at least two geometric feature values at each voxel in the plurality of voxels based on the plurality of image data values;
a mechanism for determining a colormap by assigning a color to each possible combination of the at least two geometric feature values, wherein each assigned color corresponds to a different region type within the target organ;
a mechanism for determining a display color for each voxel in the plurality of voxels based on the determined colormap; and
a mechanism for displaying, based on the determined display color for each voxel in the plurality of voxels, a color image representing the volumetric image data of the target organ.

13. The system of claim 12, further comprising:
a mechanism for identifying the at least one abnormality based on the displayed color image.

14. The system of claim 12, wherein the mechanism for determining the colormap comprises:
a mechanism for selecting a region type of the target organ;
a mechanism for determining combinations of the at least two geometric feature values that most frequently correspond to the selected region type; and
a mechanism for assigning a color to each of the determined combinations of the at least two geometric feature values that correspond to the selected region type.

15. The system of claim 14, wherein the mechanism for determining the colormap comprises:
a mechanism for obtaining simulated volume data representing the target organ; and
a mechanism for determining, based on the simulated volume data, the at least two geometric feature values for each voxel in the simulated volume data.

16. The system of claim 14, wherein the mechanism for determining the colormap comprises:
a mechanism for obtaining clinical volume data representing the target organ; and
a mechanism for determining, based on the clinical volume data, the at least two geometric feature values for each voxel in the clinical volume data.

17. The system of claim 12, wherein the mechanism for determining the at least two geometric features comprises:
a mechanism for determining, based on the plurality of image data values, a first index at each voxel, the first index being indicative of a local shape at each respective voxel; and
a mechanism for determining, based on the plurality of image data values, a second index at each voxel, the second index being indicative of a local scale at each respective voxel.

18. The system of claim 17, wherein the mechanism for determining the first index comprises:
a mechanism for determining a shape index at each voxel, the shape index being indicative of a local topology at each respective voxel.

19. The system of claim 17, wherein the mechanism for determining the second index comprises:
a mechanism for determining a curvedness index at each voxel, the curvedness index being indicative of a magnitude of a local curvature at each respective voxel.

20. The system of claim 12, further comprising:
a mechanism for detecting an abnormality region in the target organ based on calculated feature values at each voxel in the plurality of voxels,
wherein the mechanism for displaying further comprises a mechanism for displaying the detected abnormality region in a predetermined color.

21. The system of claim 12, wherein the mechanism for obtaining comprises:
a mechanism for obtaining a set of cross-sectional images of the target organ;
a mechanism for obtaining a set of voxels representing a total scanned volume from the set of cross sectional images; and
a mechanism for performing segmentation to extract, from the set of voxels representing the total scanned volume, the plurality of voxels in the volumetric image data of the target organ.

22. A system for displaying volumetric image data of a target organ as a visual aid in detecting at least one abnormality in the target organ, comprising:
an image acquisition unit configured to obtain the volumetric image data of the target organ, the volumetric image data including a plurality of voxels having a respective plurality of image data values;
a processor configured (1) to determine at least two geometric feature values at each voxel in the plurality of voxels based on the plurality of image data values, (2) to determine a colormap by assigning a color to each possible combination of the at least two geometric feature values, wherein each assigned color corresponds to a different region type within the target organ, and (3) to determine a display color for each voxel in the plurality of voxels based on the determined colormap; and
a display unit configured to display a color image representing the volumetric image data of the target organ, based on the determined display color for each voxel in the plurality of voxels.

23. A computer program product configured to store plural computer program instructions which, when executed by a computer, cause the computer perform the steps of:
obtaining the volumetric image data of the target organ, the volumetric image data including a plurality of voxels having a respective plurality of image data values;
determining at least two geometric feature values at each voxel in the plurality of voxels based on the plurality of image data values;
determining a colormap by assigning a color to each possible combination of the at least two geometric feature values, wherein each assigned color corresponds to a different region type within the target organ;
determining a display color for each voxel in the plurality of voxels based on the determined colormap; and
displaying, based on the determined display color for each voxel in the plurality of voxels, a color image representing the volumetric image data of the target organ.

24. The computer program product of claim 23, further comprising:
identifying the at least one abnormality based on the displayed color image.

25. The computer program product of claim 23, wherein the step of determining the colormap comprises:
selecting a region type of the target organ;
determining combinations of the at least two geometric feature values that most frequently correspond to the selected region type; and
assigning a color to each of the determined combinations of the at least two geometric feature values that correspond to the selected region type.

26. The computer program product of claim 25, wherein the step of determining the colormap comprises:
obtaining simulated volume data representing the target organ; and
determining, based on the simulated volume data, the at least two geometric feature values for each voxel in the simulated volume data.

27. The computer program product of claim 25, wherein the step of determining the colormap comprises:
obtaining clinical volume data representing the target organ; and
determining, based on the clinical volume data, the at least two geometric feature values for each voxel in the clinical volume data.

28. The computer program product of claim 25, further comprising:
repeating the steps of selecting a region type, determining combinations, and assigning the color for a plurality of region types of the target organ; and
assigning a color for each possible combination of the at least two geometric feature values not associated with the selected region types by interpolating the colors assigned to the selected region types.

29. The computer program product of claim 23, wherein the step of determining the at least two geometric features comprises:
determining, based on the plurality of image data values, a first index at each voxel, the first index being indicative of a local shape at each respective voxel; and
determining, based on the plurality of image data values, a second index at each voxel, the second index being indicative of a local scale at each respective voxel.

30. The computer program product of claim 29, wherein the step of determining the first index comprises:
determining a shape index at each voxel, the shape index being indicative of a local topology at each respective voxel.

31. The computer program product of claim 29, wherein the step of determining the second index comprises:
determining a curvedness index at each voxel, the curvedness index being indicative of a magnitude of a local curvature at each respective voxel.

32. The computer program product of claim 23, further comprising:
detecting an abnormality region in the target organ based on calculated feature values at each voxel in the plurality of voxels,
wherein the display step further comprises displaying the detected abnormality region in a predetermined color.

33. The computer program product of claim 23, wherein the obtaining step comprises:
obtaining a set of cross-sectional images of the target organ;
obtaining a set of voxels representing a total scanned volume from the set of cross sectional images; and
performing segmentation to extract, from the set of voxels representing the total scanned volume, the plurality of voxels in the volumetric image data of the target organ.
